(19) United States
(12) Patent Application Publication
(10) Pub. No.: US 2023/0181926 A1
(43) Pub. Date: Jun. 15, 2023

(54) DENTAL LASER TREATMENT SYSTEMS AND METHODS TO PREVENT DISCOLORATION AND AGING

(71) Applicant: Charles Holland Dresser, Wayland, MA (US)

(72) Inventors: Charles Holland Dresser, Wayland, MA (US); Nathan Monty, Shrewsbury, MA (US)

(73) Assignee: Enamel Pure, LLC, Worcester, MA (US)

(21) Appl. No.: 17/682,944

(22) Filed: Feb. 28, 2022

Related U.S. Application Data
(60) Provisional application No. 63/226,706, filed on Jul. 28, 2021.
(60) Provisional application No. 63/149,354, filed on Feb. 14, 2021.

(51) Int. Cl. A61N 5/06; A61C 19/06

(52) U.S. Cl. CPC A61N 5/062; A61C 19/066

(57) ABSTRACT

In one aspect, embodiments relate to systems and methods for preventing one or more of discoloration and attrition of dental surfaces of a patient undergoing orthodontic realignment using a clear polymer aligner. An exemplary system includes: a laser arrangement configured to generate a laser beam, an optical arrangement configured to direct the laser beam toward exposed tooth surfaces, and a laser controller configured to control the laser beam in order to heat at least a portion of the exposed tooth surfaces to affect one or more of a mechanical property and a chemical property, wherein the exposed tooth surfaces are located within a cavity of the clear polymer aligner when worn.

### CROSS-REFERENCE TO RELATED APPLICATIONS

This application claims priority benefit from Provisional Application No. 63/149,354 filed on Feb. 14, 2021, entitled “DENTAL LASER TREATMENT SYSTEM AND METHODS TO PREVENT DISCOLORATION AND AGING” and Provisional Application No. 63/226,706 filed on Jul. 28, 2021, entitled “SYNERGISTIC LASER CLEANING AND WHITENING OF TEETH” the entirety of both applications are incorporated herein by reference.

### TECHNICAL FIELD

This invention generally relates to systems and methods for preventative dental laser treatment and, more particularly but not exclusively, to systems and methods for performing specific dental laser treatments for prevention of enamel discoloration and attrition related to aging.

### BACKGROUND

Research has long showed the ability of some lasers to make dental hard tissue (e.g., enamel) less susceptible to acidic dissolution.

### SUMMARY

This summary is provided to introduce a selection of concepts in a simplified form that are further described below in the Detailed Description section. This summary is not intended to identify or exclude key features or essential features of the claimed subject matter, nor is it intended to be used as an aid in determining the scope of the claimed subject matter.

Research has long showed the ability of some lasers to make dental hard tissue (e.g., enamel) less susceptible to acidic dissolution. For example, in 1998, J. Featherstone et al. demonstrated inhibition of caries progression ranging from 40% to 85% after irradiation with infrared laser sources in an article entitled “CO₂ Laser Inhibitor of Artificial Caries-Like Lesion Progression in Dental Enamel,” published in the Journal of Dental Research and incorporated herein by reference. These results have been corroborated and repeated throughout the years. Another notable project involved researchers from University of California San Francisco and Indiana University both evaluating laser treatment for caries-inhibition in different intra-oral models. The project was documented in an article entitled “Effect of Carbon Dioxide Laser Treatment on Lesion Progression in an Intraoral Model,” published in 2001 in Proc. SPIE by J. Featherstone et al. and incorporated herein by reference.

A mechanism that is believed to contribute to this inhibition of acid dissolution of laser treated hard tissue is carbonate removal. Human dental enamel is primarily (96%-wt %) comprised of hydroxyapatite (HA). Specifically, the HA found in dental enamel is non-stoichiometric carbonate-substituted hydroxyapatite (Ca₁₀(PO₄)₆₋ₓ(OH)_{2-y})(CO₃)_{x+y}, where 0≤x≤6, 0≤y≤2, which contains trace amounts of fluoride (F), sodium (Na), magnesium (Mg), zinc (Zn) and strontium (Sr)), as reported by C. Xu et al., in an article published in 2014 in J. Material Sci., entitled “The Distribution of Carbonate in Enamel and its Correlation with Structure and Mechanical Properties,” incorporated herein by reference. Xu et al. describe that increases in carbonate content within enamel correlate with decreases in mechanical properties, for example crystallinity, modulus, and hardness. It has also been long reported that increased carbonate content within enamel correlates with an increased susceptibility to acid. For example, J. Featherstone et al. reported in “Mechanism of Laser-Induced Solubility Reduction of Dental Enamel,” first published in SPIE Proc. in 1997, and incorporated herein by reference, that carbonate removal from enamel correlates to increased resistance to caries, with complete carbonate removal correlating with the optimum resistance to caries. Caries are formed through acid dissolution. Removal of carbonate within dental enamel is achieved through elevating a temperature of the enamel.

The temperature range required for removing carbonate from dental tissue has long been taught, for example by Zuerlein et al. in an article published in 1999 in Lasers in Surgery and Medicine, entitled “Modeling the Modification Depth of Carbon Dioxide Laser-Treated Dental Enamel,” incorporated herein by reference. Zuerlein et al. found that carbonate loss began when enamel reached temperatures in excess of about 400° C. during laser irradiation, but complete carbonate removal was not achieved until the enamel reached its melting point. The melting point of dental enamel is about 1280° C. as reported by Fried et al. in an article published in 1998 in Applied Surface Science entitled “IR Laser Ablation of Dental Enamel: Mechanistic Dependence on the Primary Absorber,” incorporated herein by reference. For over 20 years it has been known to the dental research community that momentarily elevating a temperature of dental enamel to a temperature in a range between about 400° C. and about 1300° C. will reduce carbonate content and increase the enamel's resistance to acid (e.g., caries and erosion).

Additional research has shown that preventative dental laser treatment can be improved upon with application of a fluoride treatment following laser treatment. For example, referring to “Non-Destructive Assessment of Inhibition of Demineralization in Dental Enamel Irradiated by a λ=9.3-μm CO₂ Laser at Ablative Irradiation Intensities with PS-OCT,” published in Lasers in Surgery and Medicine in 2008, incorporated herein by reference, A. Can et al. present a statistically significant improvement in inhibition of demineralization of dental enamel for bovine enamel surfaces treated with both laser and fluoride over bovine enamel surfaces treated with laser alone. While the results of the scientific research have shown great promise for over 20 years, commercialization and adoption of this technology has not occurred anywhere in the world.

A commercial impediment to the wider adoption of this technology is slow adoption of new technologies in dentistry and the concomitant modest-to-low level of enthusiasm for investment and commercialization in high tech dental products. A reason that explains the relatively slow adoption of new technologies in dentistry is the fact that most dentists run their own practices. The dentist, as the owner of the dental practice, is unwilling in many cases to expend vast resources for the latest technological advancement, when those resources can be used on other (often times personal) expenses. Additionally, most technological advances require a change in workflow for the dentist. As the owner of the practice, the dentist is not often compelled to change the way she works (i.e., workflow), unless she decides to do so. A desire on the part of the dentist to not fix what isn't broken explains the slow adoption of many high technology dental products. The slow adoption of new dental technologies is also recognized in the area of professional investment. For example, although there are many professional investment groups that focus on medical devices, there are none in the United States that focus on dental devices. A mixed cause-and-result of this environment is that few new high-tech solutions reach and penetrate the dental market.

This commercial impediment is amplified by the reality that few people really prioritize their oral health. J. P. Morgan famously quipped that “A man always has two reasons for doing anything: a good reason and the real reason.” So, it is with oral health. Seldom is oral health the real reason, people take care of their teeth. Rory Sutherland, in Alchemy, published in 2019, extrapolates that the huge benefits in oral health attained by fluoride toothpaste were achieved, not because individuals valued oral health, but because they valued good smelling breath.
“A large part of why we clean our teeth I would argue is actually fear of bad breath and vanity, in the sense that would you clean your teeth after a meal at lunchtime when you're out at a restaurant? Almost certainly not. Would you clean your teeth before a date, or first thing in the morning before you go to work? Almost certainly yes. Further bit of evidence. 95% of the world's toothpaste seems to be flavored with mint, which doesn't make sense from a dental health perspective, but makes a hell of a lot of sense from a bad breath perspective.”
   It would seem that the most important oral health activity (brushing one's teeth) is motivated by both a good reason—improved oral health and a real reason—improved smelling breath. While science shows without equivocation that a good reason exists for preventive dental laser treatment, there has been no real reason for the dental market to embrace it.

Systems and methods for preventative dental laser treatment have been known to science for decades. However, the known state-of-the-art (including all of the above-mentioned references) fail to teach a way for the treatments to be made attractive to a dental market that is notoriously slow to adopt high tech solutions and a larger public whose interest in oral health is largely confined to fleeting moments sitting in the dentist's chair. In order for dental patients to benefit from decades of scientific breakthroughs in preventative dental laser treatments, laser systems and methods must be developed that are adopted by dental offices and desirable to the dental market.

Preventative laser treatment technology has been known by researchers for almost 30 years to increase caries-resistance of treated teeth up to tenfold.^{i ii iii} But the technology has never successfully been commercialized or improved the quality of life for a single dental patient (outside of clinical studies). The applicant believes the reasons for this are, in large part, outlined above and generally relate to motivations of industry stakeholders (e.g., dentists, patients, and businessmen) and to the dental industry environment in general. To bring this promising technology to market, aspects of the present invention relate not only to new technical variations for methods and systems of treatment, but applications of this technology to treat teeth in a manner never before attempted, but expected to be widely popular. At least for this reason, embodiments of the present invention relate to slowing the effects of aging (e.g., attrition and discoloration) on teeth by treating a large proportion (e.g., greater than 30%, greater than 50%, or greater than 80%) of exposed enamel surfaces with a dental laser. Just as Rory Sutherland has pointed out that many people are seemingly motivated to brush their teeth in order to have good smelling breath and appreciate the oral benefits of brushing secondarily, the applicant submits that many patients will elect dental laser treatment to slow the effects of dental aging and benefit from the known anticavity benefits of laser treatment secondarily.

As mentioned above, high-tech solutions are viewed as slow to be adopted in the dental market and professional investors eschew the opportunity to invest in high-tech dental solutions. However, an exception exists in the aesthetic dental market. Countless laser, white light, and UV whitening devices have been developed and entered into the dental market in the past 10 years. Although, the performance of these systems is modest and in some cases the claims of these systems are exaggerated, their presence in the market demonstrates an acceptance for high-tech dental solutions that promise aesthetically pleasing outcomes. University research exclusively describes preventative CO₂ laser treatment as an anti-cavity procedure, which is applied to small at-risk portions of teeth (e.g., pits and fissures). However, embodiments of the present invention relate to systems and methods that can be used to prevent enamel attrition and discoloration over all portions of teeth, thereby effectively slowing the “natural” aging process.

As a person ages the enamel on her teeth is lost to attrition. The causes of enamel loss (i.e., attrition) are varied and include decay, erosion, wear, abrasion, and abfraction. Additionally, as teeth age they are typically stained through discoloration. Dental enamel unlike other tissues does not regenerate and once gone, cannot be replaced except by artificial materials like fillings, crowns, and veneers. Commonly, the thick healthy enamel of youth is lost as a person ages and her teeth begin to appear thin, frail, worn, and discolored. Certain exemplary embodiments, described in detail herein, slow the rate of aging for teeth.

Commonly, at every dental cleaning the patient is treated with scaling of the dental hard tissue with metal picks (e.g., scalers and explorers), as well as dental prophylaxis. The reason for these procedures is, in part, to remove biofilm (e.g., tartar and plaque) that builds up on teeth. Although most people typically do not enjoy the dental visit on a good day, many patients especially dislike the descaling procedure; and, dental prophylaxis is a polishing step that removes an outer layer of the irreplaceable enamel. For at least this reason, exemplary embodiments described herein selectively remove biofilm from the teeth with touchless, sensationless laser procedure that removes no enamel. In some cases, the teeth after laser treatment are exposed, allowing for the direct application of synergistic treatments to the exposed enamel, for example aesthetic treatments such as whitening.

In order for the systems and methods described herein to fit within current dental operatory workflows, certain exemplary embodiments described herein enable treatments that can be performed in mere minutes (e.g., no more than: 1 min., 3 min., 5 min., 10 min., or 30 min.) by a dental hygienist or other trained clinician. Some versions described herein enable treatments to be performed in place of dental prophylaxis or descaling, as the teeth are cleaned from biofilm, plaque, and tartar during laser treatment. As a result, the present invention allows dental offices to benefit from the scientific advances of preventative dental laser treatments, without major workflow changes, and ideally few or no changes to the dentist's practice (which is commonly focused on restorative treatments).

Clear orthodontic aligners have gained in popularity since being developed over 20 years ago. These aligners are made of a clear plastic that fully covers each tooth and apply forces to reposition teeth (just as conventional braces do). A series of clear aligners are typically made that are intended to be used sequentially with the final clear aligner repositioning the teeth to their final (desired) location. A problem with clear aligners is that they completely cover the teeth surfaces with a clear plastic and prevent pellicle formation on the surface of the enamel. The pellicle usually forms on teeth from the presence of saliva. The pellicle is a protein film that forms directly on the enamel through selective binding of glycoproteins in the saliva. The pellicle protects the enamel from acid and staining particles. Under the clear aligners, teeth are relatively unexposed to saliva and the salivary pellicle does not form well, leaving the teeth unprotected. Bacteria, staining particles, and acids (often trapped) under the aligners affect the enamel unencumbered by a protective pellicle. As a result of this lack of protection, some users of clear aligners find that their teeth erode and discolor during realignment with clear aligners. A post-alignment whitening process can help address the discoloration, but the enamel lost to erosion during alignment will never be regained.

Clear aligners have changed the practice of orthodontics in the last 20 years, but have also introduced a new problem. Teeth underneath clear aligners are not fully protected by a salivary pellicle and, as a result, are more vulnerable to acidic erosion and pigmentary staining. Certain exemplary embodiments described herein protect teeth with a preventative dental laser treatment during realignment with clear alignments. After undergoing treatments, according to certain exemplary embodiments, the enamel has an increased resistance to acid and staining and is better protected during alignment.

In one aspect, embodiments relate to a method for slowing attrition of dental enamel, commonly associated with aging, on teeth of a patient at risk of enamel attrition. The method includes periodically performing a laser treatment on a plurality of exposed tooth surfaces on a plurality of the patient's teeth. The laser treatment, includes generating, using a laser arrangement, a laser beam; directing, using an optical arrangement, the laser beam toward a first exposed tooth surface; controlling, using a laser controller, the laser beam in order to heat at least a portion of the first exposed tooth surface to improve mechanical properties and slow attrition of the first exposed tooth surface; directing, using the optical arrangement, the laser beam toward a second exposed tooth surface; and, controlling, using the laser controller, the laser beam in order to heat at least a portion of the second exposed tooth surface to improve mechanical properties and slow attrition of the second exposed tooth surface.

In some embodiments of the method, the laser treatment is performed at least once every 5 years.

In some embodiments of the method, the patient is at risk of enamel attrition through one or more of erosion, wear, decay, abfraction, abrasion, and aging.

In some embodiments of the method, the first exposed tooth surface and the second exposed tooth surface include two or more of surfaces from a list of dental surfaces, including: a buccal surface, a facial surface, a palatal surface, a lingual surface, an occlusal surface, an interproximal surface, a mesial surface, an aesthetic surface, and a distal surface.

In some embodiments of the method, the laser beam has a wavelength within a range of between about 8 and about 11 micrometers and an average fluence within a range of between about 0.1 and about 10.0 J/cm².

In some embodiments of the method, the laser beam has a wavelength with an absorption coefficient in enamel of no more than 7,000 cm⁻¹.

In some embodiments of the method, the laser treatment has an average power no greater than 2 W and a duration of treatment no greater than 600 seconds; and therefore, the laser treatment delivers no more than 1,200 J of energy.

In some embodiments of the method, the method additionally includes: first determining the patient to be at risk of enamel attrition. In some versions, determining the patient to be at risk of enamel attrition includes a step of determining a presence of a factor believed to increase the patient's rate of enamel attrition. In some cases, the factor believed to increase the patient's rate of enamel attrition includes one or more of a current enamel thickness, the patient's diet, bruxism, and the patient's age. In some alternative versions, determining the patient to be at risk of enamel attrition includes a step of determining the patient to have a low tolerance for enamel attrition associated with aging.

In some embodiments of the method, the laser treatment includes a step of forming purified hydroxyapatite upon the exposed tooth surfaces to a depth of at least 1 micrometer. In some versions, the depth is at least 5 micrometers. In some other versions, the step of forming purified hydroxyapatite includes sublimating carbonate within an unpurified hydroxyapatite. Forming purified hydroxyapatite can be understood as converting Ca₁₀(PO₄)_{6-y}(CO₃)_{z}(OH)₂ to Ca₁₀(PO₄)₆(OH)₂ and CO₃.

In some embodiments of the method, at least one of the first exposed surface and the second exposed surface comprise an aesthetic surface. In some versions, the aesthetic surface is a surface that is visible when the patient smiles.

In another aspect, some embodiments relate to a system for slowing attrition of dental enamel, commonly associated with aging, on teeth of a patient at risk of enamel attrition. The system includes a scheduling system, configured to schedule the patient for a laser treatment on a plurality of exposed tooth surfaces on a plurality of the patient's teeth, and a laser treatment system. The laser treatment system includes a laser arrangement, configured to generate a laser beam, an optical arrangement, configured to direct a laser beam an exposed tooth surface, and a laser controller, configured to control the laser beam in order to heat the exposed tooth surface to improve mechanical properties and slow attrition of the exposed tooth surface.

In some embodiments of the system, the scheduling system is configured to schedule the patient for a laser treatment at least once every 5 years.

In some embodiments of the system, the patient is at risk of enamel attrition through one or more of erosion, wear, decay, abfraction, abrasion, and aging.

In some embodiments of the system, the exposed tooth surface comprises one or more of surfaces from a list of surfaces, including: a buccal surface, a facial surface, a palatal surface, a lingual surface, an occlusal surface, an interproximal surface, a mesial surface, an aesthetic surface, and a distal surface.

In some embodiments of the system. the laser beam has a wavelength within a range of about 8 and about 11 micrometers and an average fluence within a range of about 0.1 and about 10.0 J/cm².

In some embodiments of the system, the laser beam has a wavelength with an absorption coefficient in enamel of no more than 7,000 cm⁻¹.

In some embodiments of the system, the laser treatment has an average power no greater than 2 W and a duration of treatment no greater than 600 seconds; and therefore the laser treatment delivers no more than 1,200 J of energy.

In some embodiments of the system, the system additionally includes an annotation system configured to document a determination that the patient is at risk of enamel attrition. In some versions, the annotation system is further configured to document a presence of a factor believed to increase the patient's rate of enamel attrition. In some cases, the factor believed to increase the patient's rate of enamel attrition includes one or more of a current enamel thickness, the patient's diet, bruxism, and the patient's age. In an alternative version, the annotation system is further configured to document a patient's tolerance level for enamel attrition associated with aging.

In some embodiments of the system, the laser system is configured to form purified hydroxyapatite upon the exposed tooth surfaces to a depth of at least 1 micrometer. In some versions, the depth is at least 5 micrometers. In some additional versions, the laser system is configured to form purified hydroxyapatite by sublimating carbonate within an unpurified hydroxyapatite. Formation of purified hydroxyapatite can be understood as converting Ca₁₀(PO₄)_{6-y} (CO₃)_{z}(OH)₂ to Ca₁₀(PO₄)₆(OH)₂ and CO₃.

In some embodiments of the system, the exposed surface comprises an aesthetic surface. In some versions, the aesthetic surface comprises a surface that is visible when the patient smiles.

In yet another aspect, embodiments relate to a method for a synergist dental treatment, including, performing a laser treatment on exposed tooth surfaces on a plurality of the patient's teeth and applying a composition to the exposed tooth surfaces. The laser treatment includes generating, using a laser arrangement, a laser beam; directing, using an optical arrangement, the laser beam toward the exposed tooth surfaces; and, controlling, using a laser controller, the laser beam to remove a pellicle from the exposed tooth surfaces. Applying the composition to the exposed tooth surfaces is performed such that the composition directly wets the enamel of the exposed tooth surfaces, which have had the pellicle removed.

In some embodiments of the method, the composition includes one or more of a fluoride agent, a remineralization agent, and a whitening agent.

In some embodiments of the method, the laser beam is controlled to heat the exposed tooth surface to a temperature in a range of between about 100° C. and about 400° C. In some versions, the exposed tooth surfaces do not experience a change in mechanical or chemical properties, as a result of being heated by the controlled laser beam.

In some embodiments of the method, the method additionally includes detecting a presence of the pellicle on the exposed tooth surfaces; and, performing the laser treatment on the exposed tooth surfaces until an absence of the pellicle is detected on the exposed tooth surfaces. In some versions, detecting the presence of the pellicle on the exposed tooth surfaces is performed by measuring reflectance of a radiation.

In some embodiments of the method, the laser beam is controlled to heat the exposed tooth surface to a temperature in a range of between about 400° C. and about 1500° C. In some versions, the exposed tooth surfaces experience a change in mechanical or chemical properties, as a result of being heated by the controlled laser beam.

In some embodiments of the method, the method additionally includes detecting a presence of carbonate within the exposed tooth surfaces; and, performing the laser treatment on the exposed tooth surfaces until a reduction of carbonate is detected within the exposed tooth surfaces. In some versions, the method additionally includes detecting a first absorbance of a first radiation within the enamel surface; detecting a second absorbance of a second radiation within the enamel surface; and, relating the first absorbance to the second absorbance. In some cases, the first radiation has a first wavenumber range overlapping with 960 cm⁻¹ and the second radiation has a second wavenumber range overlapping with 1070 cm⁻¹.

In some embodiments of the method, the composition has a dosage that is less than a dosage used for treatment with the pellicle present.

In some embodiments of the method, the controller is configured to control one or more of pulse duration, peak power, repetition rate, scan speed, scan location, average power, and pulse energy.

In yet another aspect, embodiments relate to a system for a synergist dental treatment including a laser system configured to perform a laser treatment on exposed tooth surfaces on a plurality of the patient's teeth and a composition configured to be applied to the exposed tooth surfaces. Wherein, the laser system includes: a laser arrangement configured to generate a laser beam; an optical arrangement configured to direct the laser beam toward the exposed tooth surfaces; and, a laser controller configured to control the laser beam to remove a pellicle from the exposed tooth surfaces. The composition when applied to the exposed tooth surfaces, directly wets the enamel of the exposed tooth surfaces, which have had the pellicle removed.

In some embodiments of the system, the composition comprises at least one of a fluoride agent, a remineralization agent, and a whitening agent.

In some embodiments of the system, the laser controller is further configured to control the laser beam to heat the exposed tooth surface to a temperature in a range of between about 100° C. and about 400° C. In some versions, wherein the exposed tooth surfaces do not experience a change in mechanical or chemical properties, as a result of being heated by the laser beam.

In some embodiments of the system, the system additionally includes a detector, configured to detect a presence of the pellicle on the exposed tooth surfaces; and, the laser controller is configured to perform the laser treatment on the exposed tooth surfaces until an absence of the pellicle is detected on the exposed tooth surfaces. In some versions, the detector is configured to detect the presence of the pellicle on the exposed tooth surfaces by measuring reflectance of a radiation.

In some embodiments of the system, the laser controller is configured to control the laser beam to heat the exposed tooth surfaces to a temperature in a range of between about 400° C. and about 1500° C. In some versions, the exposed tooth surfaces experience a change in mechanical or chemical properties, as a result of being heated by the laser beam.

In some embodiments of the system, the system additionally includes a detector configured to detect a presence of carbonate within the exposed tooth surfaces; and, the laser controller is configured to perform the laser treatment on the exposed tooth surfaces until a reduction of carbonate is detected within the exposed tooth surfaces.

In some versions, the detector is further configured to: detect a first absorbance of a first radiation within the enamel surface; and detect a second absorbance of a second radiation within the enamel surface; and, relate the first absorbance to the second absorbance. In some cases, the first radiation has a first wavenumber range overlapping with 960 cm⁻¹ and the second radiation has a second wavenumber range overlapping with 1070 cm⁻¹.

In some embodiments of the system, the composition has a dosage that is less than a dosage used for treatment with the pellicle present.

In some embodiments of the system, the laser controller is configured to control one or more of pulse duration, peak power, repetition rate, scan speed, scan location, average power, and pulse energy.

In still another aspect, embodiments relate to a method for preventing one or more of discoloration and attrition of dental surfaces of a patient undergoing orthodontic realignment using a clear polymer aligner, including performing a laser treatment on exposed tooth surfaces on a plurality of the patient's teeth. The laser treatment includes generating, using a laser arrangement, a laser beam; directing, using an optical arrangement, the laser beam toward the exposed tooth surfaces; and, controlling, using a laser controller, the laser beam in order to heat at least a portion of the exposed tooth surfaces to affect one or more of a mechanical property and a chemical property. Wherein, the exposed tooth surfaces are located within a cavity of the clear polymer aligner when worn.

In some embodiments of the method, the mechanical property is one or more of: crystallinity, hardness, and modulus. In some versions, the laser beam affects an increase in crystallinity of the exposed tooth surfaces, thereby slowing discoloration.

In some embodiments of the method, the chemical property is one or more of: solubility to one or more acids, carbonate content, crystal shape, and crystal size. In some versions, the laser beam affects a decrease of solubility to one or more acids, thereby slowing attrition.

In some embodiments of the method, the method additionally includes applying a composition to the exposed tooth surfaces. In some versions, the composition comprises one or more of a fluoride agent, a remineralization agent, a desensitization agent, and a whitening agent.

In some embodiments of the method, the exposed tooth surfaces include an aesthetic surface.

In some embodiments of the method, the method is performed prior to application of the clear polymer aligners.

In some embodiments of the method, the method is performed during or after application of the clear polymer aligners.

In some embodiments of the method, the method is repeated after at least 1 month.

In still another aspect, embodiments relate to a system for preventing one or more of discoloration and attrition of dental surfaces of a patient undergoing orthodontic realignment using a clear polymer aligner. The system includes: a laser arrangement configured to generate a laser beam, an optical arrangement configured to direct the laser beam toward exposed tooth surfaces, and a laser controller configured to control the laser beam in order to heat at least a portion of the exposed tooth surfaces to affect one or more of a mechanical property and a chemical property, wherein the exposed tooth surfaces are located within a cavity of the clear polymer aligner when worn.

In some embodiments of the system, the mechanical property is one or more of: crystallinity, hardness, and modulus. In some versions, the laser beam affects an increase in crystallinity of the exposed tooth surfaces, thereby slowing discoloration.

In some embodiments of the system, the chemical property is one or more of: solubility to one or more acids, carbonate content, crystal shape, and crystal size. In some versions, the laser beam affects a decrease of solubility to one or more acids, thereby slowing attrition.

In some embodiments of the system, additionally include a composition configured to be applied to the exposed tooth surfaces. In some versions, the composition includes one or more of a fluoride agent, a remineralization agent, a desensitization agent, and a whitening agent.

In some embodiments of the system, the exposed tooth surfaces include an aesthetic surfaces.

In some embodiments of the system, the system is configured to perform the laser treatment prior to application of the clear polymer aligners.

In some embodiments of the system, the system is configured to perform the laser treatment during or after application of the clear polymer aligners.

In some embodiments of the system, the system is configured to repeat laser treatment after at least 3 months.

Any combination and permutation of embodiments is envisioned. Other objects and features will become apparent from the following detailed description considered in conjunction with the accompanying drawings. It is to be understood, however, that the drawings are designed as an illustration only and not as a definition of the limits of the present disclosure.

### BRIEF DESCRIPTION OF DRAWINGS

Non-limiting and non-exhaustive embodiments of the invention are described with reference to the following figures, wherein like reference numerals refer to like parts throughout the various views unless otherwise specified.

FIG. 1 illustrates an upper arch of teeth experiencing attrition, wear, thinning, increased porosity, and staining, according to certain exemplary embodiments;

FIG. 2 is a flow chart showing a method of prevented dental attrition, according to certain exemplary embodiments;

FIG. 3 is a chart depicting enamel attrition over time, according to certain exemplary embodiments;

FIG. 4 is a flow chart showing a method of removing a biofilm, according to certain exemplary embodiments;

FIG. 5A is a schematic illustration of a laser beam being used to remove a biofilm on a dental surface, according to certain exemplary embodiments;

FIG. 5B is an FTIR graph of carbonated hydroxyapatite;

FIG. 5C is a schematic illustration of illumination and detection device for detecting effective treatment, according to certain exemplary embodiments;

FIG. 5D is a schematic illustration of detected light analysis system for detecting effective treatment, according to certain exemplary embodiments;

FIG. 6 is a flow chart showing a method for preventing attrition and/or staining of dental tissue undergoing alignment with clear aligners, according to certain exemplary embodiments;

FIG. 7 is a schematic illustration of a dental arch and its corresponding clear aligner, according to certain exemplary embodiments;

FIG. 8 is an illustration of a system for dental laser treatment, according to certain exemplary embodiments;

FIG. 9 is an image of a bisected human molar with a ground flat facet undergoing in vitro testing, according to a certain exemplary embodiment;

FIG. 10 is an FTIR graph showing spectra from human molars, before and after treatment, according to certain exemplary embodiments;

FIG. 11 are two representative images showing acid erosion of a human molar with and without laser treatment, according to certain exemplary embodiments; and,

FIG. 12 is a composite micrograph showing a bisected human molar with a ground flat facet after having undergone laser treatment, according to certain exemplary embodiments.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, a complete software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment.

Some portions of the description that follow are presented in terms of symbolic representations of operations on non-transient signals stored within a computer memory. These descriptions and representations are used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each may be coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

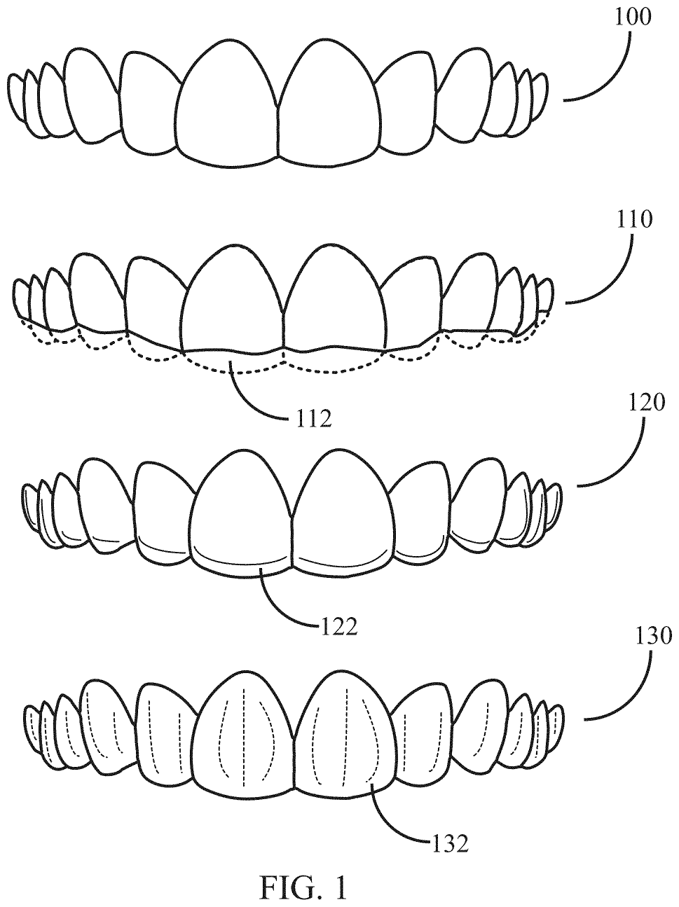

FIG. 1 illustrates an upper arch of teeth as it experiences different effects commonly associated with aging. A first arch 100 is shown in a pristine (e.g., unaged) state free from attrition, decay, or staining. A second arch 110 is shown, which has experienced attrition. Specifically, the second arch 110 has experienced wear. The teeth within the second arch 110 have a profile 112 along the biting surface (i.e., occlusal surface) that is inset from an unworn profile of teeth (as indicated by a dashed line). Although tooth wear is considered a normal part of aging, accelerated wear is considered pathological and slowing tooth wear is considered advantageous, even in non-pathological cases. Wear can be exacerbated by bruxism (i.e., grinding), clenching, a rough-textured or hard diet, developmental defects, and a misaligned bite (e.g., teeth opposing one another in an edge-to-edge manner). Wear is generally caused by physical contact of the enamel, for example by tooth-to-tooth contact, wearing away the biting surfaces. Other forms of dental aging also occur.

A third arch 120 is shown, in which teeth of the third arch have experienced enamel thinning. Thinning enamel 122 is shown for example on the occlusal surfaces of the front teeth and appears as a shear translucent section of enamel, in FIG. 1. Dental enamel thinning is often caused by enamel attrition by way of acid erosion. Erosion is technically referred to in dentistry as acid-mediated dissolution of dental hard tissue wherein the acid is not bacteria-borne. Dental erosion may be extrinsic, when the acid originates from outside the body, such as from food and drinks. Dental erosion may alternatively be intrinsic, when the acid originates from within the body, such as from stomach acid reaching the mouth, for example by way of acid reflux or bulimia. Again, enamel erosion may be considered either physiological or pathological depending upon the rate, but slower rates of enamel erosion are preferred, as slower erosion rates slow the loss of enamel and the aging of the teeth. Attrition, abrasion, wear, and erosion are factors commonly associated with aging of teeth. This factors often occur simultaneously and contribute synergistically to the aging of teeth. For example, enamel, after having been exposed to an acidic challenge (erosion), will be mechanically weakened and more likely to wear away (through tooth-to-tooth contact) or be abraded away (for example with a toothbrush). Another form of attrition which can result from bruxism is abfraction. During abfraction lateral forces applied to the teeth by grinding introduce cyclical bending moments and shear forces within the tooth. Teeth are not “designed for” this off-axis loadings and the cyclical a nature of these loadings ultimately cause enamel loss through mechanical attrition. Unlike wear that also occurs with bruxism, attrition by way of abfraction typically occurs on the non-biting surfaces of the teeth and may present like enamel thinning 120.

A fourth arch 130 is shown, in which teeth of the fourth arch 130 have experienced an increase in porosity and staining 132. Aging is commonly associated with dental discoloration. Extrinsic tooth staining is affected by numerous factors including enamel defects, salivary composition, salivary flow, poor oral hygiene, exposure to extrinsic staining agents and intrinsic physiological changes to the patient. Surface staining (i.e., discoloration of an outer surface of the teeth) is exacerbated by increased tooth surface roughness. For example, tobacco stains common among users of tobacco (e.g., cigarettes and smokeless tobacco) have been found to form at rates that are not correlative with the amount of tobacco consumed by the tobacco-user. Instead, tobacco stains form more prevalently on teeth of tobacco-users who have rougher tooth surfaces, regardless of how much tobacco they use. Pronounced enamel roughening, such as pitting, allows surface staining agents to adhere better (within the pits), discoloring the surface with their presence. Additionally, with increased adhesion, the staining agents remain proximal the enamel for longer durations and the staining agents ultimately begin to penetrate the enamel forming internal stains. Once discoloration appears within the tooth, bleaching agents (i.e., oxidizing agents), such as hydrogen peroxide, are used to remove the stains. Removal of surface stains is commonly achieved through polishing the surface of the enamel. For example, 30 seconds of dental prophylaxis removes on average about 3 micrometers of enamel. In cases of more pronounced surface staining, microabrasion and/or acid etching (for example, with hydrochloric acid) is used to remove up 200 micrometers of enamel and smooth the tooth surface. Unlike dental attrition, dental discoloration can be restored. However, in some cases (e.g., surface stains), addressing the discoloration will result in further removal of the irreplaceable enamel.

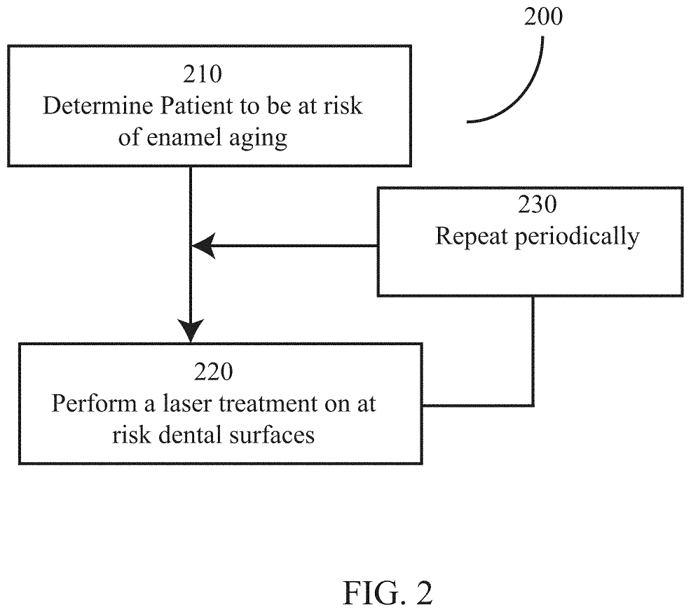

Referring now to FIG. 2, a flowchart 200 is depicted for performing a therapy in order to slow the effects of aging on teeth. First, a patient is determined to be at risk from effects of aging 210, for example enamel attrition or discoloration. Determination of a patient to be at risk of enamel aging 210 is commonly made by a dental professional (e.g., dentists or dental hygienist). Alternatively, the determination of the patient to be at risk of enamel aging 210 may be made by the patient herself, or others (e.g., a non-dental professional) close to the patient (such as, family members). A number of factors are typically considered while determining if the patient is at risk of enamel aging 210. Generally, factors can be related to the patient's rate of enamel aging (i.e., how quickly the patient is expected to either discolor or lose her dental enamel to attrition?) or the patient's tolerance to enamel aging (i.e., how important is it to the patient that her teeth remain unaged?).

Factors relating to the rate of enamel aging include a current condition of the patient's enamel, an age of the patient, a dental history of the patient, salivary production of the patient, and behaviors or habits of the patient. Determination in some cases can be made based in part upon a current condition of the patient's teeth. For example, has the patient lost a lot of enamel already given her age? In some cases, determination of a patient's risk level for enamel aging is made based in part upon her behavior. For example, in some circumstances patients that are bruxers (i.e., patients who grind their teeth) are considered to be at increased risk of enamel aging. Additionally, patients who take medications (e.g., certain blood pressure medications) that reduce saliva production are considered, in some cases, to be at increased risk of enamel aging. Patients who seldom visit the dentist's office for dental cleanings, in some cases are considered to be at an increased risk to enamel aging. Patients who consume many acidic beverages (e.g., soda, coffee, or wine) are considered in some cases to be at an increased risk of enamel aging. Patients who habitually place foreign objects in their mouth (e.g., keys, pens, or bottle caps) are in some cases considered to be at an increased risk of enamel aging. Patients who eat a hard diet are in some cases considered to be at an increased risk of enamel aging. The aforementioned are exemplary factors relating to a patient's risk for an increased rate of enamel aging. Any number of additional factors are also conceivable and are not included for brevity. In some cases, the determination that the patient is at risk to enamel aging 210 is informed by the patient's low-tolerance for aged teeth.

As described above, there are many (perhaps countless) factors that can affect a patient's rate of enamel aging. Additionally, each patient will have a different perspective on the rate at which her teeth age. Some patients will feel comfortable with their teeth aging at a physiologically “natural” rate, so long as the rate of enamel aging is not approaching pathological. Other patients will wish that their teeth remain unaged from when they were in their twenties. An individual patient, therefore, may be determined to be at risk of enamel aging even though there is no reason to believe that her rate of enamel aging is above average. A patient, in some cases, is determined to be at risk of enamel aging because the patient is desirous to slow the rate of enamel aging.

Next, a laser treatment is performed on the patient's enamel surfaces. In some cases, the treatment is performed on a number of surfaces determined to be at risk of enamel aging. In some cases, the treatment is performed especially on aesthetic enamel surfaces (i.e., enamel surfaces that are visible when the patient smiles fully). Alternatively, in some cases, the treatment is performed on most or nearly all (e.g., greater than 50% or greater than 80%) of enamel surfaces.

The laser treatment begins by generating a laser beam. The laser beam is typically generated using a laser source. Exemplary laser sources include: CO₂ lasers having a wavelength between 9 μm and 11 μm, fiber lasers, diode pumped solid state lasers (DPSS), Q-switched solid-state lasers (e.g., third harmonic Nd:YAG lasers having a wavelength of about 355 nm), Excimer lasers, and diode lasers. Commonly the laser beam has a wavelength that is well absorbed (e.g., has a wavelength having an absorption coefficient greater than 1 cm⁻¹, 100 cm⁻¹, or 1,000 cm⁻¹) by a dental hard tissue. The laser beam is then directed toward a number of surfaces of the dental tissue. In some embodiments, the laser beam is directed into an intra-oral cavity using a beam delivery system. The laser beam is often directed within the intra-oral cavity using a hand piece. In some embodiments, the laser beam is converged, using a focus optic, as it is directed toward the dental hard tissue, such that it comes to a focal region proximal the surface of the dental hard tissue. Exemplary focus optics include lenses (e.g., Zinc Selenide Plano-Convex lenses having an effective focal length of 200 mm) and parabolic mirrors. In some embodiments, the laser beam is scanned as it is directed toward the surface of the dental hard tissue by a beam scanning system. Exemplary beam scanning systems include Risley prisms, spinning polygon mirrors, voice coil scanners (e.g., Part No. MR-15-30 from Optotune of Dietikon, Switzerland), galvanometers (e.g., Lightning II 2-axis scan head from Cambridge Technology of Bedford, Mass., U.S.A.), and a gantry with a translating focus optic. Scanning methods related to dental laser systems are described in U.S. Pat. No. 9,408,673 by N. Monty et al., incorporated herein by reference.

In some embodiments, a parameter of the laser beam is controlled to affect treatment. Typically, the parameter of the laser beam is controlled in order to heat a portion of the surface of the dental hard tissue to a temperature within a range, for example between about 400° C. and about 1300° C. Exemplary laser parameters include pulse energy, pulse duration, peak power, average power, repetition rate, wavelength, duty cycle, laser focal region size, laser focal region location, and laser focal region scan speed. During laser treatment a laser beam is generated and directed toward a surface of dental hard tissue. Typically, the laser beam is pulsed at a prescribed repetition rate and has a certain pulse duration. Alternatively, pulses can be delivered on demand, and the pulse duration can vary (for example, to control heating of the surface of the dental hard tissue). As a result of the irradiation of the surface, a temperature of the surface rises typically to within a range (e.g., between 400° C. and 1300° C.) momentarily (e.g., during a duration of the laser pulse) and cools back to a normal temperature range (e.g., within a range of 20° C. and 60° C.). As a result of the momentary temperature rise biological materials previously near or adhered to the surface of the dental hard tissue (e.g., pellicle, bio-film, calculus, and tartar) are at least partially removed or denatured. In some embodiments, this removal of biological materials substantially cleans the teeth and the laser treatment replaces other tooth cleaning procedures typically performed during a dental check-up (e.g., scaling and polishing). Additionally, as described above, heating the surface of the dental hard tissue removes impurities (e.g., carbonate) from the dental hard tissue and makes the dental hard tissue less-susceptible to acid dissolution (e.g., demineralization). An exemplary laser energy dosage delivered during a single treatment does not exceed an average power of about 2 W, a treatment time of about 600 seconds, and therefore does not deliver more than about 1200 J of laser energy to the oral cavity. In some embodiments, the laser treatment is performed after other treatments during a dental visit. For example, in some cases the dental laser treatment is performed only after one or more of removal of plaque and tartar (with one or more manual instruments), professional flossing, and power polishing (i.e., dental prophylaxis). This order of steps in some cases is considered advantageous, as the laser treatment purifies only an outer portion (e.g., 2 μm thick) of the dental enamel and some dental cleaning treatments can remove a portion of dental enamel (e.g., power polishing), potentially removing the enamel which has just been purified.

In order to perform effective treatment, the enamel surface needs to have its temperature raised momentarily to within an elevated range (e.g., about 400° C. to about 1500° C.). As described throughout, elevating the temperature of enamel changes the chemical composition of hydroxyapatite within the enamel. Dental enamel comprises 96% (wt %) hydroxyapatite, 3% water, and 1% organic molecules (lipids and proteins). Specifically, dental enamel comprises 96% calcium-deficient carbonated hydroxyapatite (CAP), with a chemical formula approximated by Ca₁₀₋ₓNaₓ(PO₄)_{6-y}(CO₃)_{z}(OH)₂₋ᵤFᵤ. The ideal chemical formula for hydroxyapatite (HAP), by comparison, is approximated as Ca₁₀(PO₄)₆(OH)₂. The calcium deficiency of dental enamel is shown by the x in Ca₁₀₋ₓ. Some of the calcium is replaced by metals, such as sodium, magnesium, and potassium. These metals together total about 1% of enamel. Some of the OH molecules in dental enamel are replaced by F. But, the major difference between CAP and HAP comes with the presence of carbonate. Carbonate comprises between about 2 and about 5% (wt %) of dental enamel. The presence of carbonate within the hydroxyapatite structure disturbs a crystal lattice of the CAP, changing the size and shape of the unit crystal form and resulting in different mechanical and chemical properties between CAP and HAP. Increased carbonate content in enamel correlates with increases in susceptibility to acid and inversely correlates with crystallinity, hardness, and modulus (i.e., stiffness). Said another way the purer HAP erodes (through acid dissolution), wears (through mechanical means), and ages more slowly, compared to CAP.

As has been described in literature, including the Co-owned Int. Patent Appl. No. PCT/US21/15567, entitled “Preventative Dental Hard Tissue Laser Treatment Systems, Methods, and Computer-Readable Media”, by C. Dresser et al., incorporated herein by reference, carbonate can be removed from dental enamel by laser irradiation at prescribed parameters. Specifically, by using a laser source that is well absorbed (e.g., absorbance of at least 500 cm⁻¹) in dental enamel, and heating the surface of the tooth momentarily (e.g., at pulse durations that are no greater than 100× a thermal relaxation time) to a temperature of at least about 400° C., carbonate is driven (e.g., sublimated) from the enamel.

Next, the flowchart 200 shows that the treatment is to be repeated periodically 230. This is because, the laser treatment typically only affects an outer portion (e.g., thickness no more than about 500 μm, 100 μm, 50 μm, 10 μm, 5 μm, or 2 μm) of the treated surface. This outer portion possesses improved mechanical properties and results in a slowing of the processes associated with enamel aging. However, the outer treated portion is finite and will eventually succumb over time. For this reason, it is necessary to repeat treatment periodically 230 to ensure lasting anti-aging results. In some cases, it is advantageous to repeat treatment at least once every 10, 5, 3, 2, 1, or 0.5 years. The literature, which describes the vast potential of laser treatment to prevent caries, fails to suggest that periodic retreatment may be necessary and instead tends to assume that a one-time treatment will suffice for most patients.

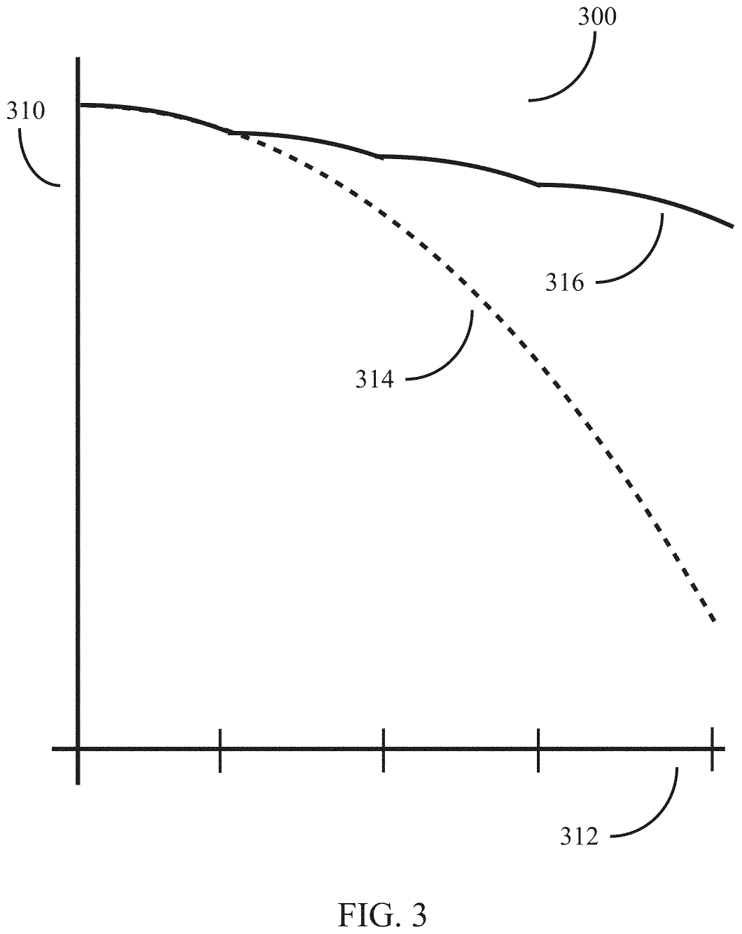

Advantages of periodic retreatment is illustrated by way of a graph 300 in FIG. 3. The graph 300 illustrates enamel thickness (for example, in millimeters) along a vertical axis 310 and time (for example, in years) along a horizontal axis. Enamel thickness over time for a one-time treated patient is shown with a dashed line 314; and, enamel thickness over time for a periodically treated patient is shown with a solid line 316. Both the one-time treated and periodically retreated patients are shown in the graph 300 to have been treated at time=0. Over time, the one-time treated patient experiences a decline in enamel thickness that accelerates as time progresses and the multi-factorial synergistic effects of wear, abrasion, attrition, erosion, increased porosity, and decay all work together to thin enamel at ever faster rates. By comparison the patient who undergoes periodic retreatments 316, is continually refreshing her teeth's resistance to aging (e.g., attrition, wear, erosion, decay, etc.). The periodically retreated patient 316 undergoes treatments at 4 substantially regular intervals (e.g., 6 month time intervals, 1 year time intervals, 2 year time intervals, 5 year time intervals, or 10 year time intervals) after time=0. As a result, there exists a dramatic difference in enamel thickness between the one-time treated patient 314 and the periodically retreated patient 316 at the end of the fourth time interval.

In some embodiments, laser treatment removes one or more of a biofilm, calculus, tartar, and pellicle. Currently, during conventional dental cleanings, a dental scalar is used to remove plaque, calculus, and tartar from the surface of teeth, for example between teeth and near the gumline. Many patients especially dislike this process during the dental cleaning, when the metal scaler scrapes and picks at their teeth. Instead, in some embodiments, a non-contacting sensationless (e.g., touchless, noiseless, smell-less, etc.) laser treatment is used to remove calculus, tartar, or plaque from dental hard tissue surfaces, including enamel, dentine, and cementum.

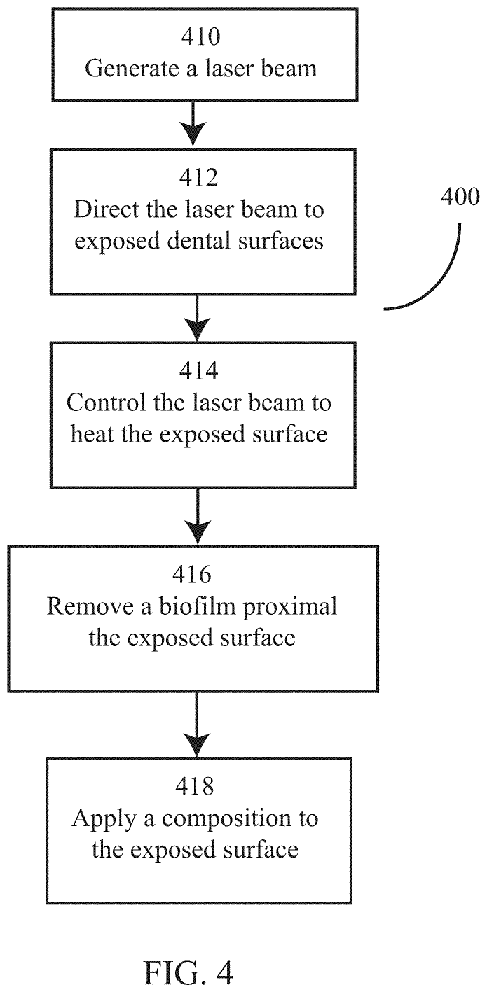

Referring to FIG. 4, a flowchart 400 is presented that describes a method of removing a biofilm from a dental surface using a laser treatment. First, a laser beam is generated 410. The laser beam typically has a wavelength in a range between about 8 and 12 micrometers (e.g., about 9.3 μm, about 9.6 μm, or 10.6 μm). The laser beam is typically pulsed and has a pulse duration, that is about a thermal relaxation time of the enamel and laser beam (e.g., the pulse duration is no more than 10 to 100 times greater than the thermal relaxation time). In some embodiments, the laser beam is pulsed at a repetition rate that has a period (i.e., inverse of the repetition rate) that is greater than the thermal relaxation time (e.g., greater than the thermal relaxation time, greater than 10 times the thermal relaxation time, or greater than 100 times the thermal relaxation time).

Thermal relaxation time is defined, in some cases, to represent an estimated amount of time required for thermal diffusion to reduce temperature in a layer of dental tissue, having a certain thickness, by approximately one half. Commonly, the thickness of the layer of dental tissue is taken to be an optical penetration depth, which is approximated as an inverse of the absorbance coefficient of the laser radiation in dental tissue, or:

$$X ( λ ) = \frac{1}{{μ}_{a} ( λ )}$$

where, X(λ) is the optical penetration depth as a function of wavelength, λ is wavelength of the laser, and μₐ(λ) is the absorption coefficient of dental tissue at the laser wavelength. The thermal relaxation time, or time for a temperature required for tissue at a certain depth to reach about 84% of a surface temperature, is approximated as:

$$t = \frac{{x}^{2}}{4 K}$$

where, t is the thermal relaxation time, x is the depth of the location of the tissue, and K is a thermal diffusivity of the tissue (e.g., enamel, dentine, or cementum). In some cases, it is appropriate to calculate an axial (depth orientated) thermal relaxation time, as described above and (by using the optical penetration depth as x). Alternatively, it is appropriate to calculate a radial thermal relaxation time that represents an amount of time for tissue radially displaced from the laser beam to heat as a result of pulsed laser cooling (by using a width of the laser beam as x). In many cases, the laser beam width is larger than the optical penetration depth and as a result the shorter of the two thermal relaxation times (axial and radial) is the axial thermal relaxation time. Thermal diffusivity is given as:

$$K = \frac{k}{ρ {c}_{p}}$$

where, K is the thermal diffusivity (for example, in units of m²/s, k is thermal conductivity (for example, in units of W/[mK]), ρ is density (for example, in units of kg/m³), and cₚ is specific heat capacity (for example, in units of J/[kgK]). Exemplary thermal parameters for dental enamel include, a density of about 2.9 g/cm³, a specific heat capacity of about 0.75 J/(g° C.), a thermal conductivity of about 9.2×10⁻³ W/(cm° C.), and a thermal diffusivity of about 0.0042 cm²/s. Exemplary thermal relaxation times for dental enamel with a 10.6 micron and 9.6 micron laser are about 1 μs and about 90 μs, respectively.

The laser beam is directed to exposed dental surfaces 412. Exposed dental surfaces comprise all tooth surfaces in the mouth, including buccal surfaces, facial surfaces, palatal surfaces, lingual surfaces, occlusal surfaces, interproximal surfaces, mesial surfaces, and distal surfaces. Generally, all tooth surfaces in the mouth have a salivary pellicle formed upon them. This pellicle layer (i.e., biofilm) is formed from proteins and glycoproteins in saliva. Under normal oral conditions, the pellicle layer protects the underlying tissue surface (i.e., enamel, dentin, or cementum). For example, the pellicle protects the dental tissue from direct exposure to acids, such as those formed by bacteria or those ingested by the patient. The pellicle is also normally colonized by bacteria (e.g., gram positive aerobic cocci, such as Streptococcus sanguinis, Streptococcus mutans, and Lactobacilli). Because the pellicle covers and protects the dental surfaces, it also prevents dental surfaces from being directly exposed to any number of compositions that are being employed to bring about a desired effect. For example, some tooth whitening procedures first instruct the patient to brush her teeth with a weak acid formulation to break down the pellicle, before applying the whitening (e.g., hydrogen peroxide) composition to whiten her teeth. Brushing one's teeth with acid is not a daily routine that most would consider healthy or anti-aging for teeth, but it increases the speed of effective whitening treatment by allowing the enamel to be directly wetted by the whitening composition.

The laser beam, as it is directed to the exposed dental surface, is typically better absorbed by the underlying dental surface (e.g., enamel) than by the pellicle layer. For example, an absorption coefficient of a CO₂ laser beam in enamel is 8,000 cm⁻¹, 5,500 cm⁻¹, and 825 cm⁻¹ for 9.3 μm, 9.6 μm, and 10.6 μm wavelengths respectively.^{iv v} For the same 9.3 μm, 9.6 μm, and 10.6 μm wavelengths the absorption coefficient in 100% water is about 600 cm⁻¹ and in 4% water is about 30 cm⁻¹.^{vi} Depending upon a hydration level of the salivary pellicle a nominal to moderate amount of absorption of laser radiation will occur within the pellicle. Conversely, a moderate to very high level of laser radiation occurs within the enamel. In cases of high laser absorption (e.g., absorption coefficient greater than 600 cm⁻¹) in enamel and low laser absorption (e.g., absorption coefficient less than 600 cm⁻¹) in the pellicle, most of the laser energy is absorbed in a surface of the enamel. The laser energy absorbed in the outer surface of the enamel typically occurs in such a narrow width (the width of the laser beam) (e.g., less than 2 mm, less than 1 mm, less than 0.5 mm or less than 0.25 mm) and at such a thin depth (approximated by an optical penetration depth, which is an inverse of the absorption coefficient) (e.g., less than 0.2 mm, less than 0.1 mm, less than 0.02 mm, less than 0.01 mm, less than 0.005 mm, or less than 0.002 mm) that a small amount of energy (e.g., less than 100 mJ, less than 50 mJ, less than 20 mJ, less than 10 mJ, less than 5 mJ, or less than 2 mJ) raises a temperature of the surface of the enamel significantly (e.g., greater than 50° C., greater than 100° C., greater than 200° C., greater than 500° C., greater than 700° C., or greater than 1000° C.) momentarily (e.g., less than 10 ms, less than 1 ms, less than 0.5 ms, or less than 0.1 ms). As a result of this momentary rise of enamel surface temperature, the pellicle layer is removed (e.g., vaporized, sublimated, ablated, or denatured). Unlike other forms of removing the pellicle layer (e.g., dental prophylaxis, acid etching, or abrasion), laser treatment does not risk removal of the enamel, but substantially only the pellicle layer, along with any plaque, tartar or surface contaminants (i.e., biofilm), is removed. In some embodiments, the enamel is raised to a temperature within a first range between about 100° C. and about 400° C. In this first range, the salivary pellicle is substantially removed, but the enamel does not experience any substantial improvements to its mechanical properties (e.g., removal of carbonate, increased crystallinity, increased modulus [i.e., stiffness], increased resistance to acid or increased hardness). In some cases, heating the enamel surface within this first range is advantageous as the pellicle is removed, while the underlying enamel remains receptive to topical compositions (e.g., whiting agents, remineralization agents, and fluoride treatments). Alternatively, in some embodiments, the enamel is raised to a temperature within a second range between about 400° C. and about 1500° C. When heated to a temperature within this second range, the surface of the enamel has the pellicle layer removed and also experiences improvements to its mechanical properties (e.g., removal of carbonate, increased crystallinity, increased modulus [i.e., stiffness], increased resistance to acid, or increased hardness). Heating of enamel to a temperature within this range removes carbonate impurities from within the enamel surface.^{vii} A lack of carbonate impurities within enamel (i.e., hydroxyapatite) correlates with an increase in mechanical properties, such as crystallinity, modulus, hardness, and resistance to acid.^{viii ix}

In some embodiments, one or more laser parameters are controlled to control the temperature rise of the dental surface. Exemplary parameters that can be controlled to affect the temperature rise include, pulse duration, pulse energy, repetition rate, fluence, irradiance, peak power, average power, number of overlapping pulses at a given location, and time between pulses (i.e., repletion period). The fluence of the laser at the surface of the enamel is commonly selected to affect a temperature rise of dental enamel. For example, with a 9.3 micron laser and a pulse duration in a range between about 0.1 and about 100 μs, a fluence greater than about 0.5 J/cm² and less than about 5 J/cm² causes elevation of enamel surface temperature to within the 400° C. to 1500° C. range. Predictive modeling of effects of laser treatment on surface temperature rise has been found substantially accurate. For example, a nodal finite element analysis using Fourier conduction, Beer's absorption, and Newton's cooling with known parameters has been performed by the applicant. This analysis demonstrated that predictable surface temperature results are attained through use of the model. The model was verified by bench tests with multiple laser sources having peak powers ranging from about 50 W to about 1000 W. Further disclosure related to parameter selection for dental surface temperature rise and nodal-FE analysis for parameter selection is described in detail in U.S. Patent Appl. No. 62/968,910, entitled Laser Delivery of Transverse Electromagnetic Modes for Even Preventative Dental Hard Tissue Treatment, by N. Monty et al., incorporated herein by reference. Predictable temperature rise based upon known thermal and photonic constants allows for the selection and control of parameters to control temperature rise. For example, in some embodiments, a laser parameter is controlled in order to control the temperature rise of a non-enamel dental hard tissue (e.g., dentin, cementum, or osseous tissue) to a range having a lower boundary and an upper boundary. The lower boundary being selected to exceed a denaturing threshold of the biofilm (e.g., at least 50° C. or at least 100° C.). The upper boundary being selected not to exceed a tissue combustion, carbonization, incineration, or melting threshold (e.g., no more than about 200° C., no more than about 400° C., no more than about 600° C., or no more than about 1000° C.).

In some cases, removal of the biofilm completes the laser procedure. Alternatively, in some embodiments, a composition is applied directly to the surface 418, with substantially no pellicle (or biofilm) layer between the composition and the surface. In some embodiments, the direct application of the composition without an intermediary pellicle or biofilm layer improves the efficacy of the composition and, in some cases, allows a decreased dosage (e.g., concentration of active ingredient) of the composition to be used. Exemplary compositions include whiting agents, fluoride treatments, desensitizing agents, remineralization agents, sealants, composite filings, etches, wetting agents, and adhesives.

In some cases, the composition includes a fluoride composition. Fluoride treatment is known to cause fluorapatite to form in dental hard tissues. Fluorapatite is a harder mineral and more resistant to acid than is hydroxyapatite. Application of a fluoride composition after removal of the biofilm allows the fluoride to be applied directly to the dental hard tissue surface and not need to first diffuse through the protective salivary pellicle. In some cases, this direct application of the fluoride composition increases the effectiveness of the fluoride composition. Exemplary fluoride compositions include Sodium Fluoride, Stannous Fluoride, Titanium Tetrafluoride, Acidulated-Phosphate Fluoride, and Amine Fluoride. In some cases, the fluoride composition takes a form of one or more of a varnish, a gel, a foam, a liquid, and a dentifrice.

In some cases, the composition includes a whitening agent. As described above whitening agents are reduced in efficacy by the protective salivary pellicle, which prevents all of the oxidizing agents (present in the whitening agent) from reaching the underlying hard tissue. As a result, some whitening procedures call for pellicle damaging agents and procedures to be applied prior to the application of the whitening agent. Instead, the laser treatment removes the pellicle (and other biofilms if present) allowing the whitening agent to be applied directly to the dental hard tissue undergoing whitening (e.g., enamel, dentine, or cementum). In some cases, direct application of the whitening agent to the dental hard tissue allows the whitening agent to have a reduced dosage (e.g., reducing concentration or reduced quantity of whitening agent). In some embodiments, the whitening agent comprises one or more of hydrogen peroxide, carbamide peroxide, and sodium perborate.

In some cases, the composition includes a remineralization agent or a desensitizing agent. When carbonate is removed from the enamel surface it leaves behind calcium and phosphate deficient hydroxyapatite. Application of a remineralization agent after laser treatment, in some cases, introduces a calcium or phosphate rich material proximal the enamel surface allowing the calcium and phosphate deficient hydroxyapatite to uptake calcium and phosphate. In some embodiments, the remineralization agent comprises a composition, which is substantially adherent to the enamel surface, such that pellicle formation occurs over the composition (e.g., a varnish or a gel) effectively enriching the pellicle matrix with calcium and/or phosphate. In some embodiments, the remineralization agent comprises one or more of fluoride remineralization agents (see below), nonfluoride remineralization agents (e.g., Alpha tricalcium phosphate [TCP] and beta TCP [β-TCP], Amorphous calcium phosphate [ACP], casein phosphopeptide-stabilized amorphous calcium phosphate [CPP-ACP], Sodium calcium phosphosilicate [bioactive glass], Xylitol, Dicalcium phosphate dehydrate [DCPD], Nanoparticles for remineralization [e.g., Calcium fluoride nanoparticles, Calcium phosphate-based nanomaterials, NanoHydroxyapatite {NanoHAP} particles, ACP nanoparticles, Nanobioactive glass materials]), Polydopamine, proanthocyanidin [PA], Oligopeptides, Theobromine, Arginine, Self-assembling peptides, and Electric field-induced remineralization.

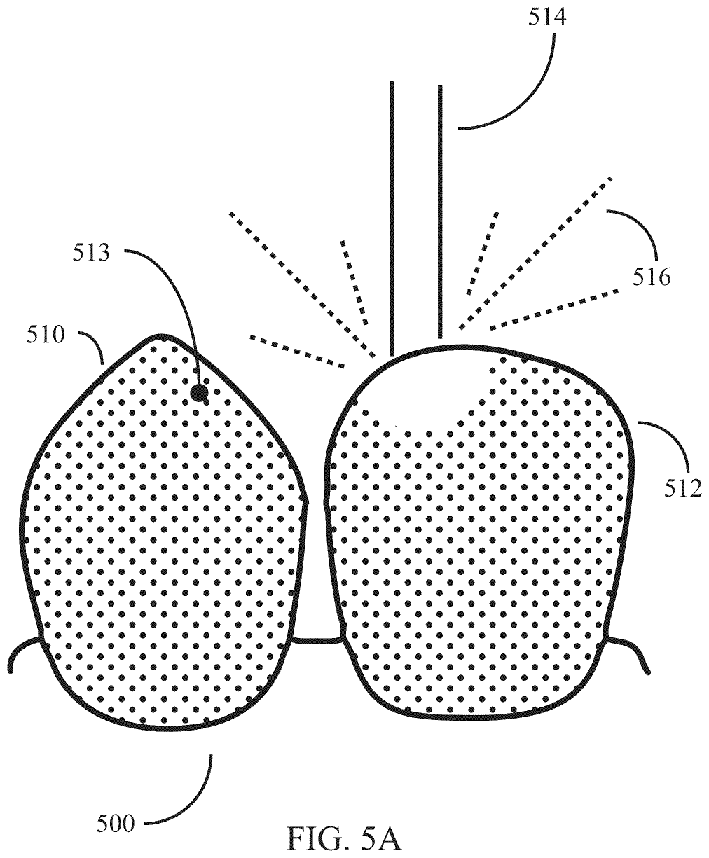

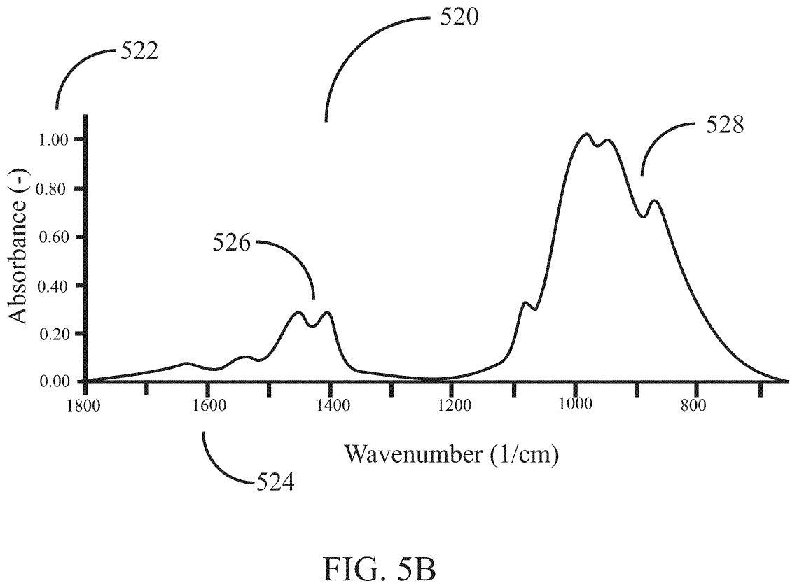

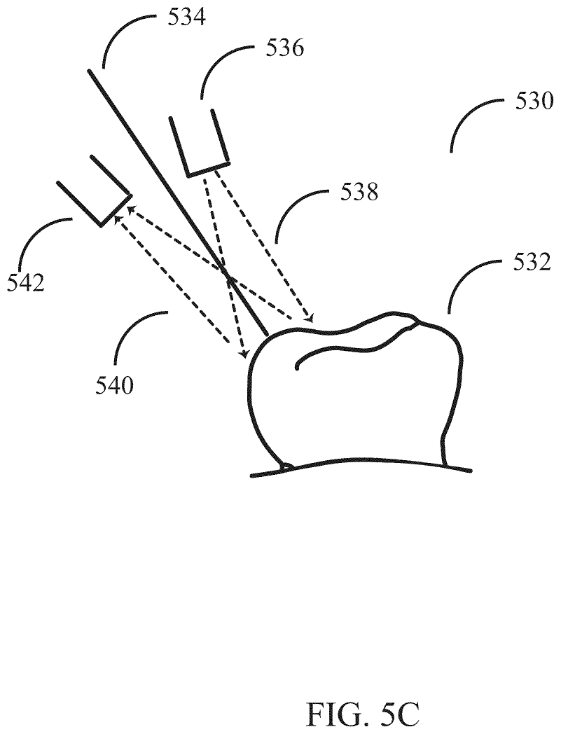

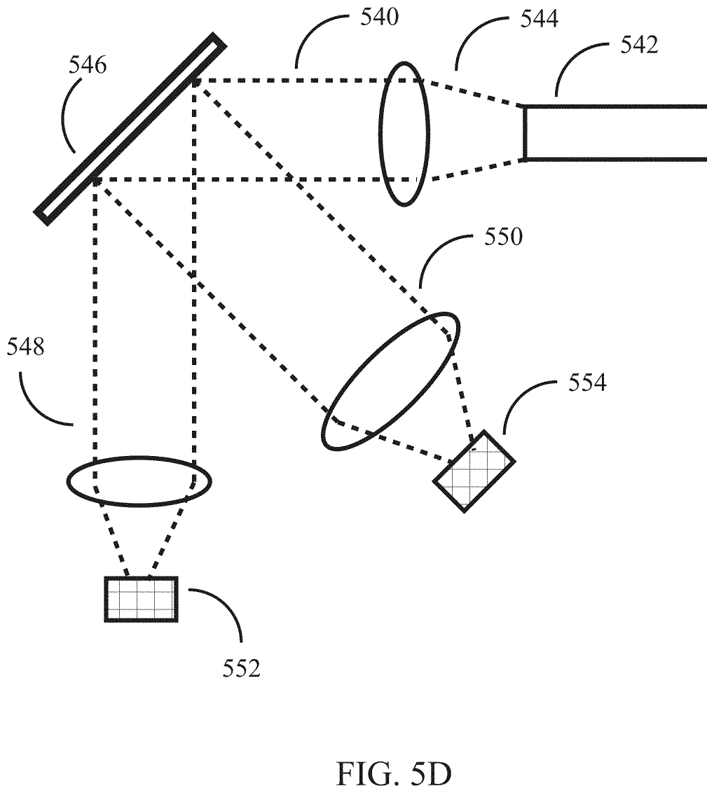

Referring to FIG. 5A, a schematic illustration of a mouth 500 having a first tooth 510 and second tooth 512. The first tooth 510 is completely covered by a biofilm 513. The second tooth 512 is shown having the biofilm 513 partially removed by a laser beam 514. The laser beam 514 is directed toward a surface of the second tooth 512. The laser beam 514 heats a portion of the surface of the second tooth 512. Heating of the surface of the second tooth causes the biofilm 513 to be removed. In some cases, the biofilm is removed by way of sublimation, vaporization, thermal denaturization, photonic denaturization, or laser ablation. In some cases, the biofilm is moderately well absorbed by the laser beam (e.g., has an absorption coefficient greater than about 100 cm⁻¹) and heating of the biofilm at least partially contributes to removal of the biofilm. The removed biofilm (e.g., biofilm particulate and/or vapors) 516 is shown being ablated away from the laser heated surface, in FIG. 5A.

Referring to FIG. 5B, an FTIR graph 520 is shown for dental enamel (i.e., carbonate hydroxyapatite). The graph 520 has absorbance, in arbitrary units, along a vertical axis 522 and wavenumber, in inverse centimeters, along a horizontal axis 524. As can be seen in the graph 520, dental enamel has two pronounced absorbance peaks in the plotted range. A carbonate peak 526 is present between about 1300 and about 1500 cm⁻¹. A phosphate peak 528 is present between about 700 and about 1200 cm⁻¹. In accordance in some embodiments, carbonate is removed on the treated surface of the enamel during laser treatment. When carbonate is removed from the surface of the enamel, the carbonate absorbance peak 526 decreases while the phosphate absorbance peak 528 remains substantially unchanged. According to some embodiments, a system is incorporated to detect effective laser treatment has been performed on the enamel surface.

FIG. 5C shows an illustration 530 of a tooth 532 undergoing a treatment with a device configured to detect when an enamel surface has been effectively treated, according to a certain exemplary embodiment. A laser beam 534 is directed to the tooth to perform the laser treatment. In addition to the laser beam 534, an illumination pathway 536 directs illumination 538 to the surface of the tooth 530. In some cases, the illumination 538 is configured to irradiate the tooth surface at a location that is at least partially overlapping with the laser beam 534. Reflected illumination 540 from the surface of the tooth (at least partially overlapping the laser beam 534) is collected by a return light path 542.

FIG. 5D illustrates a light analysis arrangement 543 configured to analyze the reflected light 540, according to a certain exemplary embodiment. The return light path 542 provides the light analysis arrangement 543, with the reflected light 540. The reflected light, in some cases, is acted upon by an optical arrangement 544 that directs the light to a spectral beamsplitter 546 (e.g., a grating or a prism). The spectral beamsplitter 546 splits the reflected light 540 into two or more light channels, each light channel being differentiated according to wavelength. In some embodiments, the reflected light 540 is split into two light channels, a first light channel 548 and a second light channel 550. The first light channel 548 is detected by a first light sensor 552. The second light channel 550 is detected by a second light sensor 554. Alternatively, the first light channel 548 and the second light channel 550 are detected by a single light sensor, such as an image digitizing sensor array (e.g., CMOS or CCD image or array sensor).

According to a certain exemplary embodiment, the first light channel 548 at least partially overlaps with a wavelength range that is representative of the carbonate peak 526 (e.g., wavenumber between about 1100 and about 1300 cm⁻¹) and the second light channel 550 at least partially overlaps with a wavelength range that is representative of the phosphate peak 528 (e.g., wavenumber between about 700 and 1200 cm⁻¹). The illumination light 538 includes wavelengths of light in both the first and second channels. In some cases, the illumination light 538 comprises broad spectrum infrared light. The illumination pathway 536, in some cases, includes a 2 to 11 micrometer wavelength light source (e.g., Thorlabs Part No. HPIR104). Broad spectrum infrared light in the illumination pathway 536 and return pathway 542 is directed by way of one or more of a free space (e.g., mirror reflected) optical arrangement, a hollow waveguide, a chalcogenide fiber optic (e.g., AsSe core-clad fiber), and polymer IR materials (e.g., Ploy-IR from Fresnel Technologies).

According to some embodiments, at least one of a presence or an absence of a biofilm (e.g., pellicle layers) is detected during treatment. For example, in some cases, the light analysis arrangement detects the presence or the absence of the biofilm upon a surface being treated, intra-treatment. For example, the presence of biofilm, in some cases, is detected by an attenuation of one or more absorbance bands commonly associated with dental enamel (e.g., the carbonate band and the phosphate band). Alternatively, the presence of biofilm, in some cases, is detected by a presence of an absorbance (or reflectance) band associated with a substance (e.g., chromophore or fluorophore) present within the biofilm (e.g., protein, glycoprotein, bacteria, carbohydrates, lipids, and water). In some cases, the absence of the biofilm is detected by a detection condition that is contrary (or contradictory) to a detection condition related to the presence of biofilm.

In some embodiments, at least one of a presence or an absence of carbonate within the dental hard tissue is detected. For example, in some cases, the light analysis arrangement detects the presence or the absence of carbonate within a surface being treated, intra-treatment. For example, the presence of carbonate, in some cases, is detected by a comparing an amount of light detected from the first return light channel 548 and the second return light channel 550. For example, the first light channel 548 and the second light channel 550 in some cases are analyzed to determine absorbance values for the carbonate peak 526 and the phosphate peak 528. The analyzed absorbance values for the carbonate peak 526 and the phosphate peak 528, in some cases, are then compared, for example ratiometrically, to estimate carbonate content within the dental tissue relative phosphate content. A ratio of carbonate content to phosphate content will decrease dramatically during certain laser treatments described herein, allowing for effective determination of treatment effectiveness intra-treatment.

According to some embodiments, dental enamel crystallinity is detected within the dental hard tissue. For example, in some cases, the light analysis arrangement detects a width of an absorbance band associated with the phosphate peak 528 and estimates crystallinity (i.e., relative crystallinity) of the dental enamel based upon this width. In some cases, as treatment is performed and carbonate is removed from the dental enamel, the crystalline structure of the enamel becomes more uniform. As a result of the increased crystallinity, the vibrational modes associated with phosphate molecules within the crystal become less varied. This decrease in variation of phosphate vibrational modes is evident through analysis of a width of the phosphate absorption band.

In some embodiments, a laser treatment is controlled (intra-treatment) based upon results of the return light analysis. For example, in some cases, the laser treatment is controlled to continue until (1) the biofilm is removed (i.e., substantially absent); (2) carbonate is removed (i.e., substantially absent) from the dental tissue; or, (3) the dental tissue increases in crystallinity. In still other cases, laser parameters are controlled intra-treatment based upon the return light analysis. For example, in one case, one or more laser parameters related to treatment fluence (i.e., pulse duration, pulse energy, or beam width) are controlled based upon a presence or an absence of one or more of biofilm, carbonate, and crystallinity. In some versions, the return light analysis returns a relative measure relating to the absence or the presence of one or more of the biofilm, the carbonate, or the crystallinity and the laser treatment is controlled based upon this relative measure.

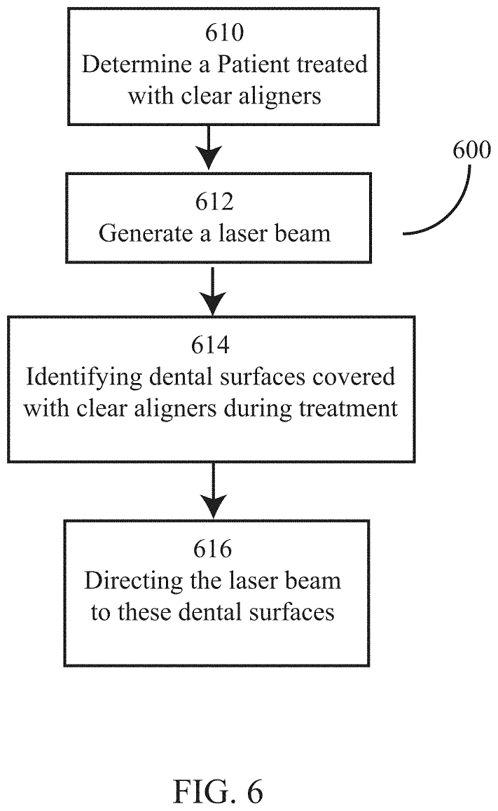

Referring now to FIG. 6, a flowchart 600 is shown describing a method for preventing enamel deterioration (e.g., attrition and discoloration) as a patient undergoes orthodontic alignment with clear aligners. For about the past 20 years, clear aligners have become a popular option for orthodontic alignment. Unlike conventional metal braces, clear aligners are nearly invisible when worn and can be removed throughout the day by the patient. Clear aligners are made from a clear polymer and they are shaped to closely match a profile of teeth undergoing realignment, with slight deviations in profile that force the teeth to be repositioned within the mouth over time. Typically, a series of multiple clear aligners are used one after another until a desired orthodontic alignment is achieved. As described above, a problem with clear aligners is that they cover substantially the entirety of the tooth surfaces within the mouth (or at least many of the tooth surfaces). As the tooth surfaces are covered by a clear polymer, they are not exposed to saliva and no salivary pellicle is substantially formed on the teeth. As described above, the salivary pellicle provides a protective barrier against destructive agents in the mouth (e.g., acids, bacteria, and staining agents). Without a fully formed salivary pellicle, teeth under the clear polymer aligners are less protected from the destructive agents and enamel deterioration occurs, for example by way of attrition (e.g., erosion, wear, and decay) or discoloration (e.g., staining). In addition to preventing the formation of a pellicle, in some cases, the clear aligners can actually trap destructive agents (e.g., bacteria, sugars, acids, staining pigments, contaminants, etc.) proximal the enamel. With the clear plastic aligners in place, the rinsing function commonly performed by saliva does not occur and these destructive agents remain close to the largely unprotected enamel until the patient removes the aligners and brushes her teeth. According to certain embodiments of the present invention, patients using clear aligners are selected for laser treatment to prevent enamel deterioration commonly associated with use of clear aligners.

According to the flowchart 600, first, a patient undergoing orthodontic alignment with clear aligners is determined 610. In some cases, the patient is currently undergoing alignment (i.e., is currently wearing clear aligners on a regular basis or as a course of therapy). Alternatively, in some cases, the patient is expected to begin orthodontic alignment in the future. Determining that the patient is using clear aligners 610, in some embodiments, is performed by one or more of a dentist, an orthodontist, a dental hygienist, another medical or dental professional, the patient herself, or another person close to the patient. In some cases, the practitioner who is prescribing the orthodontic treatment helps make the determination that the patient is now or soon will be undergoing orthodontic alignment using clear aligners.

A laser beam is then generated 612. As described throughout, in some embodiments the laser beam comprises a wavelength in a range of about 8 to 12 micrometers. In some embodiments, the laser beam is pulsed with a pulse duration not much greater than a thermal relaxation time of the enamel. Commonly, the laser beam is generated 612 with a laser source. Exemplary laser sources include carbon dioxide (CO₂) lasers, carbon monoxide (CO) lasers, excimer lasers, fiber lasers, diode pumped solid state (DPSS) lasers, and semiconductor lasers.

Dental surfaces covered by the clear aligners (when in place) are then identified 614. In many cases, the clear aligners cover all of the teeth in an arch (e.g., upper arch or lower arch). In this case, all of the teeth surfaces in the arch would be identified as being covered by the clear aligners during treatment. The laser beam is then directed to the enamel surfaces covered with clear aligners during alignment 616. The clear aligners are typically removed during treatment, as the polymer of the clear aligners, in some cases, absorbs the laser beam. For this step, the laser beam is directed to enamel surfaces, which are covered by the clear aligners when the clear aligners are in place (i.e., on the teeth). The laser beam is controlled to effect a desired treatment. For example, the laser beam in some cases heats the tissue momentarily improving the mechanical properties of the enamel. Exemplary mechanical properties that are typically improved through treatment include, crystallinity, hardness, stiffness, and resistance to acidic dissolution. The laser beam, in some embodiments, is controlled to raise a surface temperature of the enamel surfaces. Typically, one or more parameters of the radiation are controlled with a controller. An exemplary controller includes laser control boards (e.g., Maestro from LANMark Controls Inc. of Acton, Mass., U.S.A.

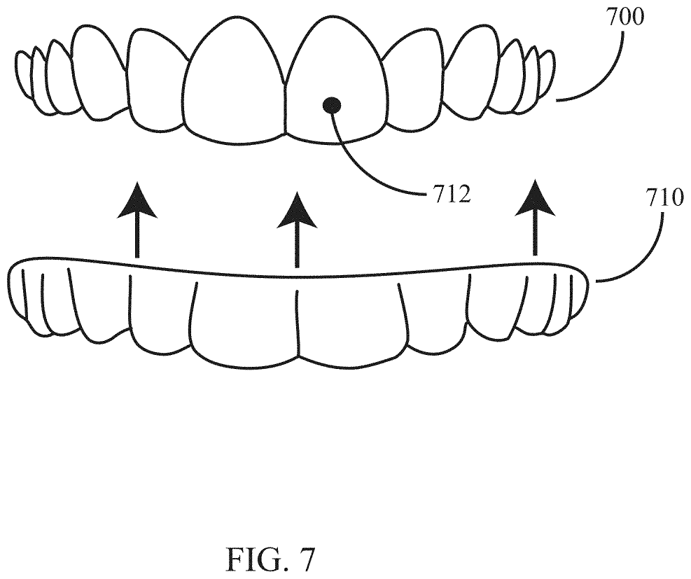

Referring to FIG. 7, an upper arch 700 is shown along with a corresponding clear aligner 710. The clear aligner is configured to attach over the upper arch. In the case shown in FIG. 7, all of the tooth surfaces of the upper arch 700 are effectively covered by the clear aligners when the clear aligners are attached. Of increased importance, in some cases, are aesthetic dental surfaces 712. These aesthetic dental surfaces 712 are those, which are visible (or prominent) when the patient smiles. The appearance of these aesthetic surfaces 712 are most valuable by many dental patients. In some embodiments, the aesthetic surfaces undergo laser treatment.

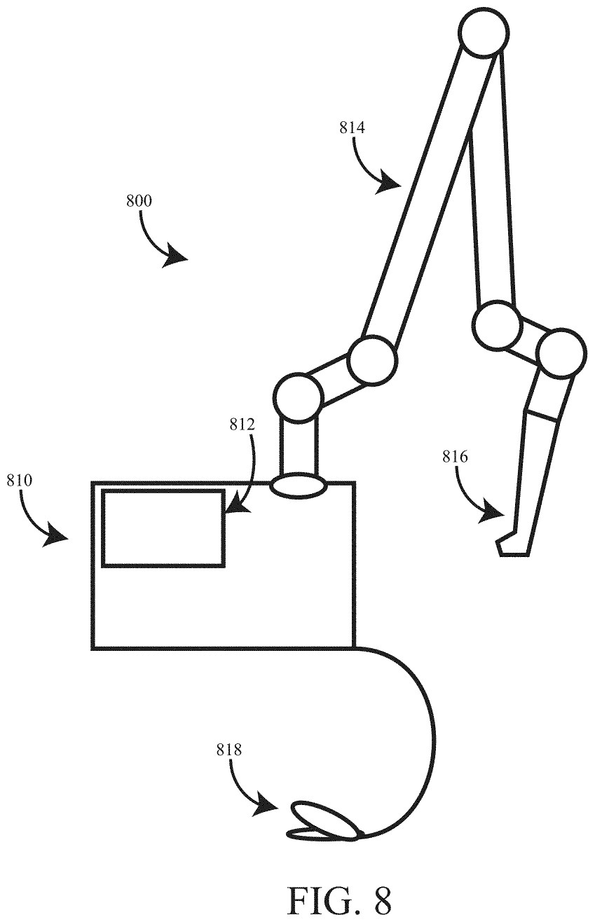

An exemplary system 800 is shown in FIG. 8. The system 800 includes a console 810. The console 810 houses components of the system 800, for example, a laser source to generate the laser beam, a direct current (DC) power supply to power the laser source, a beam shaper to shape an energy profile of the laser beam, a compressed air system to deliver compressed air for bulk cooling of dental hard tissue being treated, and a user interface 812 for user control. A beam delivery system 814 directs the laser beam to a hand piece 816. Exemplary beam delivery systems 814 include articulated arms, waveguides, and fiber optics. An exemplary articulated arm is provided by Laser Mechanisms of Novi, Mich., U.S.A. The hand piece 816 is configured to be used intra-orally (i.e., within an oral cavity). Typically, the hand piece 816 includes a focus optic (not shown) that converges the laser beam to a focal region outside of the hand piece 816. In accordance with one embodiment, the system 900 is operated with a foot pedal 818, which is configured to initiate the laser source.

In accordance with one embodiment, the system 800 is used by a clinician. First, the clinician inputs operating parameters into the user interface 812, for example by using a touch screen. Then the clinician places the hand piece 816 within a patient's mouth and directs the hand piece 816 toward dental hard tissue. For example, the clinician positions the hand piece 816 so that a focal region of the laser beam is coincident with or near (e.g., +/−1 mm, 2 mm, 3 mm, or 5 mm) a surface of a tooth. Then, the clinician activates the laser by stepping on a foot pedal 818. The clinician moves the hand piece 816 within the patient's mouth, carefully directing the focal region of the laser beam near every treatment surface of the patient's teeth.

To aid in practice of the claimed invention and parameter selection a table is provided below with exemplary ranges and nominal values for relevant parameters.

| Parameter | Min. | | Max. | | Nom. | |
|---|---|---|---|---|---|---|
| Repetition Rate | 1 | Hz | 100 | KHz | 1 | KHz |
| Pulse Energy | 1 | μJ | 10 | J | 10 | mJ |
| Focal Region Width | 1 | μm | 10 | mm | 1 | mm |
| Fluence | 0.01 | J/cm² | 1 | MJ/cm² | 1 | J/cm² |
| Wavelength | 200-500 | nm | 4000-12000 | nm | 10.6 | μm |
| Numerical Aperture (NA) | 0.00001 | | 0.5 | | 0.01 | |
| Focal length | 10 | mm | 1000 | mm | 200 | mm |
| Average Power | 1 | mW | 100 | W | 1 | W |
| Peak Power | 50 | mW | 5000 | W | 500 | W |
| Scan Speed | 0.001 | mm/S | 10 | mm/S | 100,000 | mm/S |

| | | | |
|---|---|---|---|
| Scan Location Spacing | 0 | 0.5× Focal | 10× Focal |
| | | Region Width | Region Width |

| | |
|---|---|
| Bleaching Agents | Hydrogen Peroxide, Carbamide Peroxide, Sodium Perborate |
| Remineralizing and/or | Fluorides (see below), Nonfluoride remineralizing agents (e.g., Alpha |
| Desensitizing Agents | tricalcium phosphate [TCP] and beta TCP [β-TCP], Amorphous |
| | calcium phosphate [ACP], casein phosphopeptide-stabilized |
| | amorphous calcium phosphate [CPP-ACP], Sodium calcium |
| | phosphosilicate [bioactive glass], Xylitol, Dicalcium phosphate |
| | dehydrate [DCPD], Nanoparticles for remineralization [e.g., Calcium |
| | fluoride nanoparticles, Calcium phosphate-based nanomaterials, |
| | NanoHydroxyapatite {NanoHAP} particles, ACP nanoparticles, |
| | Nanobioactive glass materials]), Polydopamine, proanthocyanidin |
| | [PA], Oligopeptides, Theobromine, Arginine, Self-assembling |
| | peptides, and Electric field-induced remineralization |
| Fluoride Agents | Sodium Fluoride, Stannous Fluoride, Titanium Tetrafluoride, |
| | Acidulated-Phosphate Fluoride, and Amine Fluoride |

### Exemplary Embodiment

Further explanation is provided below with an exemplary embodiment demonstrating effective treatment of dental surfaces to prevent deterioration common with aging.

Method—A 9.3 μm CO2 laser was used. The laser was a Luxinar OEM45ix. The laser was operated at an average power not in excess of 1 W. Output from the laser was directed by three reflectors and aligned into a galvanometer scan head. The laser was output from the galvanometer scan head and focused by focusing optics. The width of the laser focal region was determined using a 90-10 knife edge method. A knife (e.g., razor blade) was placed in front of the beam at the focal region and scanned transverse to the laser axis. A thermopile was used to measure laser power down beam from the knife edge. The knife edge location where the laser power is found to be at 10% and 90% was measured using a calibrated stage. This distance (i.e., 90-10 knife edge distance) was used to estimate the 1/e² beam width. The 1/e² beam width was then used to determine the desired pulse energy from a fluence (i.e., energy density) range known to affect treatment without melting: between about 0.5 and 1.0 J/cm^{2 x}. Pulse energy was calculated by measuring the average power using a thermopile (Ophir PN: 30A-BB-18) and dividing by an average repetition rate, determined by an oscilloscope (PicoScope 2205A) measuring the amplified signal from a fast infrared photodiode (Hamamatsu PN: C12494-011LH). A laser scan pattern was developed using ScanLab software. A laser controller controlled the laser and the galvanometers to deliver the laser beam deterministically according to the scan pattern. The scan pattern sequentially delivered individual laser pulses to individual scan locations, in order to increase the effective treatment area of the laser beam, without increasing the focal region width (which would require greater laser energy per pulse to maintain the necessary energy density [fluence]). Additionally, each sequential pulse in the scan pattern was delivered to a location, which is non-adjacent to the previous pulse, with 7 individual laser pulses elapsing between adjacent pulses. This method of spacing sequential pulses maximized the amount of time between adjacent pulses, which allowed more time for the surface to cool post laser pulse to its initial temperature before another laser pulse acting at the same (or proximal) location was delivered. The laser scan pattern width was approximately 2.5 mm wide. An effective treatment area of this size or larger allows for a clinical viable treatment speed. For example, it was estimated that the treatable surface area of teeth in the mouth is about 2500 mm², from available anatomical data^{xi}. A 2.5 mm wide laser treatment, if scanned over the teeth at a rate of 5 mm/S, will theoretically be completed in a little under four minutes.

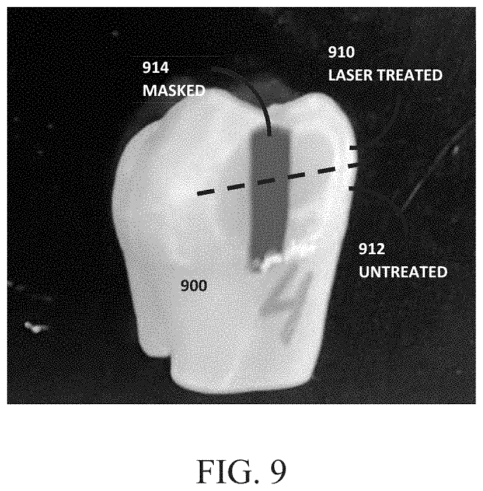

Referring to FIG. 9, testing with the exemplary embodiment was performed using bisected human molars 900 having a ground flat facet. Each flat facet was masked with copper tape, so that only a treated half 910 of the flat facet was exposed to the laser treatment. The untreated half 912 beneath the copper tape was left untreated. The facet was then treated with the laser scan pattern at a prescribed pulse duration and 1 W average power. During treatment of the facet a smear layer was removed, as evidenced by smoke (i.e., particles and vapors) visibly emanating from the surface. Likewise, overtreatment of the facet, onto the unground surface of the tooth, showed similar smoke during initial treatment which dissipated after first laser irradiation. This smoke is believed to have been a result of removal of a biofilm layer (e.g., pellicle, calculur, and/or tartar) on the unground portion of the tooth. After laser treatment, the copper tape was removed and tape residue was removed with acetone and a cotton swab. Next, a thin stripe of nail polish 914 was applied to the flat facet. The nail polish stripe was applied perpendicularly to the direction the copper tape was applied. So, the nail polish 914 covered and left exposed two areas of the facet, which comprised laser treated 910 and untreated 912 areas. The samples were then exposed to acid erosive challenges of varying durations. Acid erosive challenge parameters included a citric acid buffer, 3.2 pH, and 35° C. temperature. After the erosive challenge, the nail polish was removed with acetone and each tooth was examined with a confocal microscope (Lieca DCM8). Depth of erosion (relative to the nail polish masked surface, which was not exposed to the acid) was determined for both laser treated and untreated surfaces and compared to reveal a percent reduction in erosion. In some cases, prior to the erosive challenge, the tooth surface was analyzed using an FTIR spectrometer (Nicolet Summit with an IRSurvey microsampler attachment). An FTIR spectra of laser treated and untreated surfaces of the bisected molar facet was compared to determine percent carbonate reduction resulting from treatment. Finally, the surfaces of the teeth were examined under high-powered microscope to ensure that overheating of tooth surface (i.e., melting) did not occur, even at a microscopic level. A number of laser parameters were tested. Representative laser parameters are shown in the table below.

| | Pulse | | | | | | | |
|---|---|---|---|---|---|---|---|---|
| Sam- | Dura- | | Rep. | Pulse | Avg. | Peak | | Ero- |
| ple | tion | Power | Rate | Energy | Fluence | Fluence | FTIR | sion |
| (—) | (uS) | (W) | (Hz) | (mJ) | (J/cm2) | (J/cm2) | (Y/N) | (min) |
| 1 | | | | 2.5 | 0.74 | 1.48 | Yes | 10 |
| 2 | 16.5 | 1.04 | 481 | 2.16 | 0.64 | 1.28 | Yes | 10 |
| 3 | 16.8 | 1.05 | 448 | 2.34 | 0.69 | 1.39 | No | 5 |
| 4 | 17.4 | 1.05 | 417 | 2.52 | 0.74 | 1.49 | No | 15 |
| 5 | 16.7 | 1.03 | 481 | 2.14 | 0.63 | 1.27 | No | 5 |
| 6 | 17.3 | 1.05 | 419 | 2.51 | 0.74 | 1.48 | No | 15 |

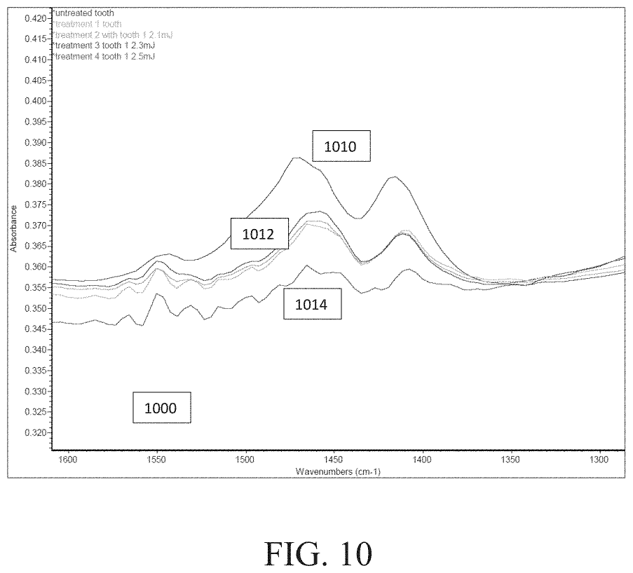

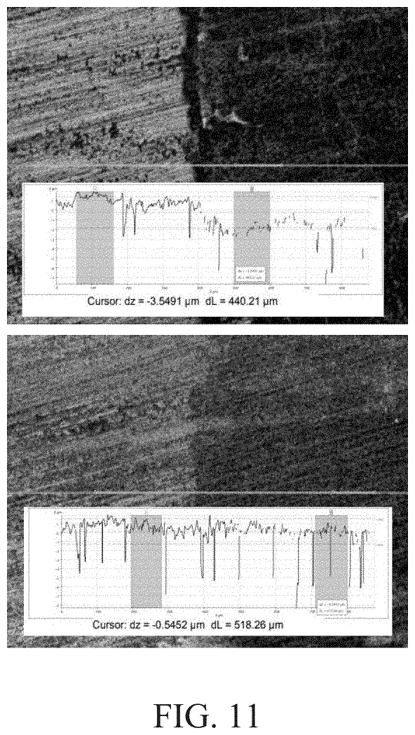

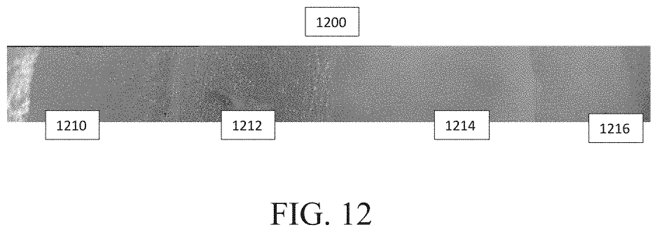

Results—Laser treatment with the exemplary embodiment was able to achieve both carbonate reduction and acid erosion resistance in human enamel. For example, 50% or more carbonate reduction correlated with a marked increase in acid demineralization resistance (e.g., 80%). It was found that most laser settings were able to achieve at least 50% carbonate reduction without introducing surface overheating. Also, some laser parameters could effectively remove all the carbonate from the surface of the tooth without surface damage. Referring to FIG. 10, FTIR spectra 1000 measured from teeth pre-treatment 1010 and post-treatment 1012, 1014 are shown. It can be seen that about 50% carbonate reduction 1012 corresponds with pulse energies of about 1.9-2.3 mJ (Avg. fluence between 0.5-0.7 J/cm²), while complete carbonate removal is achieved with pulse energies of 2.5 mJ (Avg. fluence>0.7 J/cm²). Additionally, confocal microscope profilometry revealed reduced erosion with the laser treated surfaces. FIG. 11 illustrates representative confocal measurements showing a sample experienced 3.5 μm of erosion in an untreated portion of the tooth and only 0.5 μm of erosion where the tooth hand been laser treated. This corresponds to an 86% reduction in erosion for the laser treated surface compared to the untreated surface. FIG. 12 shows a stitched composite microscopic image 1200 of ground flat facet of a human molar having four vertical stripes treated with: 0.6 J/cm2 fluence (no surface damage) 1210, 0.9 J/cm2 (melting) 1212, untreated 1214, and 0.7 J/cm2 (no surface damage) 1216. It was found, with the exemplary embodiment, that fluences of about 0.9 J/cm² cause melting of the surface, which is seen only under a microscope. However, fluence settings used for treatment (even those capable of removing all of the carbonate from the surface) can be delivered to the teeth without causing melting.

Conclusion—The exemplary embodiment was able to produce an effective treatment at a speed suitable for the clinic, without damaging the surface of the teeth. Using the 9.3 μm laser, an average fluence setting between 0.5 and 0.75 J/cm² removes carbonate, increases acid resistance, and does not damage the tooth surface. With this bench test, it was demonstrated that treatment can be performed quickly, using a large (e.g., 2.5 mm wide) scanned laser pattern and that this pattern can be arranged to prevent overtreatment (and overheating of the surface). Additionally, this treatment was demonstrated to be effective using both acid erosive tests and FTIR spectral analysis.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. For example, in some embodiments, fluoride treatment is omitted after laser treatment. Also, features described with respect to certain configurations may be combined in various other configurations. Different aspects and elements of the configurations may be combined in a similar manner. Technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims.
ⁱ Featherstone, J. D. B., Barrett-Vespone, N. A., Fried, D., Kantorowitz, Z., &. Seka, W. (1998). CO2 laser inhibition of artificial caries-like lesion progression in dental enamel. Journal of dental research, 77(6), 1397-1403.
ⁱⁱ Featherstone, J. D., & Fried, D. (2001). Fundamental Interactions of Lasers with Dental Hard Tissues. Medical laser application, 16(3), 181-194.
ⁱⁱⁱ Hsu, D. J., Darling, C. L., Lachica, M. M., & Fried, D. (2008). Nondestructive assessment of the inhibition of enamel demineralization by CO2 laser treatment using polarization sensitive optical coherence tomography. Journal of biomedical optics, 13(5), 054027.
^{iv} Featherstone, J. D., & Fried, D. (2001). Fundamental Interactions of Lasers with Dental Hard Tissues. Medical laser application, 16(3), 181-194.
^{v} Featherstone, J. D., & Fried, D. (2001). Fundamental Interactions of Lasers with Dental Hard Tissues. Medical laser application, 16(3), 181-194.
^{vi} Vitruk, P. (2014). Oral soft tissue laser ablative and coagulative efficiencies spectra. Implant Practice US, 7(6), 19-27.
^{vii} Zuerlein, M. J., Fried, D., & Featherstone, J. D. (1999). Modeling the modification depth of carbon dioxide laser-treated dental enamel. Lasers in Surgery and Medicine: The Official Journal of the American Society for Laser Medicine and Surgery, 25(4), 335-347.
^{viii} Xu, C., Reed, R., Gorski, J. P., Wang, Y., & Walker, M. P. (2012). The distribution of carbonate in enamel and its correlation with structure and mechanical properties. Journal of materials science, 47(23), 8035-8043.
^{ix} Featherstone, J. D. B., & Lussi, A. (2006). Understanding the chemistry of dental erosion. In Dental erosion (Vol. 20, pp. 66-76). Karger Publishers.
^{x} Kim, J. W., Lee, R., Chan, K. H., Jew, J. M., & Fried, D. (2017). Influence of a pulsed CO 2. laser operating at 9.4 μm on the surface morphology, reflectivity, and acid resistance of dental enamel below the threshold for melting. Journal of biomedical optics, 22(2), 028001.
^{xi} Woelfel, J. B., & Scheid, R. C. (1997). Dental anatomy. Williams & wilkins.

What is claimed is:

1. A system for preventing one or more of discoloration and attrition of dental surfaces of a patient undergoing orthodontic realignment using a clear polymer aligner, the method comprising:
a laser arrangement configured to generate a laser beam;
an optical arrangement configured to direct the laser beam toward exposed tooth surfaces; and,
a laser controller configured to control the laser beam in order to heat at least a portion of the exposed tooth surfaces to affect one or more of a mechanical property and a chemical property, wherein the exposed tooth surfaces are located within a cavity of the clear polymer aligner when worn.

2. The system of claim 1, wherein the mechanical property is one or more of crystallinity, hardness, and modulus.

3. The system of claim 2, wherein the laser beam affects an increase in crystallinity of the exposed tooth surfaces, thereby slowing discoloration.

4. The system of claim 1, wherein the chemical property is one or more of solubility to one or more acids, carbonate content, crystal shape, and crystal size.

5. The system of claim 4, wherein the laser beam affects a decrease of solubility to one or more acids, thereby slowing attrition.

6. The system of claim 1, further comprising a composition configured to be applied to the exposed tooth surfaces.

7. The system of claim 6, wherein the composition comprises one or more of a fluoride agent, a remineralization agent, a desensitization agent, and a whitening agent.

8. The system of claim 1, wherein the exposed tooth surfaces comprise an aesthetic surface.

9. The system of claim 1, wherein the system is configured to perform the laser treatment prior to application of the clear polymer aligners.

10. The system of claim 1, wherein the system is configured to perform the laser treatment during or after application of the clear polymer aligners.

11. A method for preventing one or more of discoloration and attrition of dental surfaces of a patient undergoing orthodontic realignment using a clear polymer aligner, the method comprising:
performing a laser treatment on exposed tooth surfaces on a plurality of the patient's teeth, wherein the laser treatment comprises:
generating, using a laser arrangement, a laser beam;
directing, using an optical arrangement, the laser beam toward the exposed tooth surfaces; and,
controlling, using a laser controller, the laser beam in order to heat at least a portion of the exposed tooth surfaces to affect one or more of a mechanical property and a chemical property, wherein the exposed tooth surfaces are located within a cavity of the clear polymer aligner when worn.

12. The method of claim 11, wherein the mechanical property is one or more of crystallinity, hardness, and modulus.

13. The method of claim 12, wherein the laser beam affects an increase in crystallinity of the exposed tooth surfaces, thereby slowing discoloration.

14. The method of claim 11, wherein the chemical property is one or more of solubility to one or more acids, carbonate content, crystal shape, and crystal size.

15. The method of claim 14, wherein the laser beam affects a decrease of solubility to one or more acids, thereby slowing attrition.

16. The method of claim 11, further comprising applying a composition to the exposed tooth surfaces.

17. The method of claim 16, wherein the composition comprises one or more of a fluoride agent, a remineralization agent, a desensitization agent, and a whitening agent.

18. The method of claim 11, wherein the exposed tooth surfaces comprise an aesthetic surface.

19. The method of claim 11, wherein the method is performed prior to application of the clear polymer aligners.

20. The method of claim 11, wherein the method is performed during or after application of the clear polymer aligners.
